SEQUENCE LISTING (16 sequences)

SEQ ID NO: 1 - protein, 25 aa, Homo sp. [sequence omitted]

SEQ ID NO: 2 - protein, 10 aa, Mus musculus
GYTFNGYWMH

SEQ ID NO: 3 - protein, 14 aa, synthetic construct
WVRQAPGQGLEWIG

SEQ ID NO: 4 - protein, 17 aa, Mus musculus
YINPTTGHTEYNQKFKD

SEQ ID NO: 5 - protein, 30 aa, synthetic construct
KATITADESTNTAYMELSSLRSEDTAVYYC

SEQ ID NO: 6 - protein, 12 aa, Mus musculus
ARQEYRHSWFAY

SEQ ID NO: 7 - protein, 11 aa, Homo sp.
WGQGTLVTVSS

SEQ ID NO: 8 - protein, 26 aa, synthetic construct [sequence omitted]

SEQ ID NO: 9 - protein, 11 aa, Mus musculus
QSVDYDGDTYM

SEQ ID NO: 10 - protein, 16 aa, Homo sp.
NWYQQKPGKAPKLLIY

SEQ ID NO: 11 - protein, 7 aa, Mus musculus
AASNLDS

SEQ ID NO: 12 - protein, 32 aa, Homo sp.
GVPSRFSGSGSGTDFTLTISSLQPEDFATYYC

SEQ ID NO: 13 - protein, 9 aa, Mus musculus
QQTNEDPWT

SEQ ID NO: 14 - protein, 11 aa, Homo sp.
FGQGTKVEIKR

SEQ ID NO: 15 - protein, 119 aa, synthetic construct
QVQLVQSGAEVKKPGSSVKVSCKASGYTFNGYWMHWVRQAPGQGLEWIGYINPTTGHTEYNQKFKDKATITADESTNTAYMELSSLRSEDTAVYYCARQEYRHSWFAYWGQGTLVTVSS

SEQ ID NO: 16 - protein, 112 aa, synthetic construct
DIQLTQSPSSLSASVGDRVTITCRASQSVDYDGDTYMNWYQQKPGKAPKLLIYAASNLDSGVPSRFSGSGSGTDFTLTISSLQPEDFATYYCQQTNEDPWTFGQGTKVEIKR

(19) United States
(12) Patent Application Publication
(10) Pub. No.: US 2023/0122862 A1
(43) Pub. Date: Apr. 20, 2023

(54) TREATMENT OF IGE-MEDIATED DISEASES

(71) Applicant: United BioPharma, Inc., Hsinchu County (TW)

(72) Inventors: Be-Sheng KUO, Hsinchu County (TW); Chao-Hung LI, Hsinchu County (TW); Ywan-Feng LI, Hsinchu County (TW); Shugene LYNN, Hsinchu County (TW); Chang Yi WANG, Hsinchu County (TW)

(73) Assignee: United BioPharma, Inc., Hsinchu County (TW)

(21) Appl. No.: 17/964,101

(22) Filed: Oct. 12, 2022

Related U.S. Application Data
(60) Provisional application No. 63/254,729, filed on Oct. 12, 2021.

(51) Int. Cl. C07K 16/42; A61P 37/08; A61P 11/06

(52) U.S. Cl. CPC C07K 16/4291; A61P 37/08; A61P 11/06; A61K 2039/505

(57) ABSTRACT

The present invention relates to the treatment of IgE mediated diseases using an anti-IgE antibody. In particular, the anti-IgE antibody is a multifunctional antibody against IgE, which neutralizes IgE and inhibits IgE synthesis. Specifically, the treatment of the present invention is effective in providing a rapid and/or sustained suppression of disease symptoms.

### RELATED APPLICATION

This application claims priority to U.S. Provisional Application No. 63/254,729, filed on Oct. 12, 2021, the content of which is hereby incorporated by reference in its entirety.

### REFERENCE TO AN ELECTRONIC SEQUENCE LISTING

The contents of the electronic sequence listing (UB0001WO-seg list.xml; Size: 14,884 bytes; and Date of Creation: Oct. 7, 2022) is herein incorporated by reference in its entirety.

### TECHNOLOGY FIELD

The present invention relates to the treatment of IgE-mediated diseases using an anti-IgE antibody. In particular, the anti-IgE antibody is a multifunctional antibody against IgE, which neutralizes IgE and inhibits IgE synthesis. Specifically, the treatment of the present invention is effective in providing a rapid and/or sustained suppression of disease symptoms.

### BACKGROUND OF THE INVENTION

There remains an unmet medical need for a diverse array of allergic diseases, of which the combined disease prevalence has profoundly increased to affect more than 25% of the world population, causing global health threat and mounting economic burden^{1,2}. The allergic (atopic) diseases such as food allergy, atopic dermatitis, asthma and allergic rhinitis could be of inter-related and sometimes referred to as “Atopic March,”³⁻⁵ which may initiate from childhood and is largely IgE-mediated. Non-allergic diseases could also be IgE-associated, which do not involve an immune response to a foreign antigen, particularly inflammatory skin diseases, including chronic spontaneous urticaria (CSU).

The functions of IgE are hinged upon interactions of its Fc region (Cε2-Cε4) with two principal receptors: the high-affinity FcεRI (K_{D}, approximately 10⁻¹⁰-10⁻¹¹ M)⁶, expressed mainly on mast cells and basophils, responsible for allergic hypersensitivity and inflammation; and the low-affinity, monomeric CD23 (FcεRII) (K_{D}, approximately 10⁻⁶-10⁻⁷ M)⁷, expressed mainly on B cells, involved in the regulation of IgE synthesis and IgE clearance and a host of other immunological functions⁸⁻¹¹. CD23 on cellular surfaces commonly exists as a homotrimer. The IgE binding affinity to the free trimeric CD23, or interaction in the form of IgE-immune complexes (IgE-IC) with CD23, could yield an avidity strength (K_{D}, 10⁻⁹-10⁻¹⁰ M) approaching that for the IgE-FcεRI interaction¹².

Given reported beneficial protective effects of IgE against worms and cancers^{13,14}, IgE, due to its harmful effector functions manifesting in allergic symptoms, is generally perceived as physiologically dispensable and as a legitimate, safe target for drug development. Omalizumab is the only anti-IgE antibody approved up to date, used for a few IgE-associated diseases, and is restricted to serve as a third-line add-on therapeutic for moderate-to-severe persistent allergic asthma (2003), chronic spontaneous urticaria (CSU, 2014), and nasal polyps (2020)¹⁵. New anti-IgE biologicals have been pursued preclinically and clinicallyl^{6,17}. However, alternative IgE-targeting antibodies (anti-Cε) explored and remained viable in late-phase clinical trials have been ligelizumab (QGE031) only^{18,19}.

As the standard front-line treatment for CSU, small molecule drugs (e.g., anti-histamine) or cyclic peptide drugs (e.g., cyclosporine) are usually administered with high dosing frequencies. Daily medication with pills or capsules for long weeks, however, oftentimes leads to incompliance that makes their disease difficult to control. When frontline treatments fail to work, patients would need to resort to biologics, i.e., the only-approved omalizumab or ligelizumab which has been tested on a pivotal phase 3 trial but eventually failed in a late-stage development. While these two anti-IgE monoclonal antibodies are administered every 4 weeks.

In this art, there is still a need for efficacious and improved anti-IgE antibodies for treating IgE-mediated diseases.

### SUMMARY OF THE INVENTION

The present invention is at least based on the finding that a multifunctional anti-IgE antibody which neutralizes IgE and inhibits IgE synthesis provides efficacious and improved effects in treating IgE-mediated diseases. In particular, the anti-IgE antibody inhibits or blocks CD23-mediated downregulation of IgE production. Specifically, the treatment using such a multifunctional anti-IgE antibody provides rapid and/or sustained symptomatic relief, and a less frequent dosing is required that increases patient comfort and convenience.

In one aspect, the present invention provides a method for treating an IgE-mediated disease, comprising administering to a subject in need thereof an anti-IgE antibody, wherein the antibody is a multifunctional antibody which neutralizes IgE and inhibits IgE synthesis. Specifically, the method of the present invention provides rapid and/or sustained symptomatic relief in the subject.

In some embodiments, the antibody binds to free IgE, membrane-bound IgE on B lymphocytes, and/or IgE bound by CD23, but not to IgE bound by FIERI on mast cells.

In some embodiments, the antibody binds in a free form with CD23-bound IgE and in an IgE-complex form with CD23.

In some embodiments, the antibody is an antigen-binding fragment thereof.

In some embodiments, the antibody is humanized.

In some embodiments, the antibody or antigen-binding fragment comprises

(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and

(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13.

In some embodiments, the VH comprises the amino acid sequence of SEQ ID NO: 15.

In some embodiments, the VL comprises the amino acid sequence of SEQ ID NO: 16.

In some embodiments, the symptomatic relief includes IgE reduction, alleviation in itch and/or decrease of hive counts.

In some embodiments, the symptomatic relief is sustained for 2 to 14 weeks or longer after administration.

In some embodiments, the symptomatic relief occurs within one week or faster after administration.

In some embodiments, the antibody is administered every 2 to 14 weeks or less frequently.

In some embodiments, the antibody is administered every 4 weeks or less frequently.

In some embodiments, the antibody is administered every 12 to 24 weeks.

In some embodiments, the antibody is administered in a dosage of 0.1 to 10 mg per 1 kilogram of body weight of the subject.

In some embodiments, the antibody is included in a composition in an entire dose and is administered to the subject in a single dose.

In some embodiments, the antibody is administered by either intravenous or subcutaneous injection.

In some embodiments, the IgE-mediated disease is allergic asthma, allergic rhinitis, atopic dermatitis, food allergy, chronic spontaneous (idiopathic) urticaria, chronic rhinosinusitis, systemic mastocytosis, cutaneous mastocytosis, allergic bronchopulmonary aspergillosis, recurrent idiopathic angioedema, or eosinophil-associated gastrointestinal disorder.

The present invention also provides an anti-IgE antibody as described herein or a pharmaceutical composition comprising the same for use in treating an anti-IgE mediated disease. Further disclosed is the use of an anti-IgE antibody as described herein for manufacturing a medicament for treating an anti-IgE mediated disease.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows kinetic analyses of IgE binding to the anti-IgE monoclonal antibodies UB-221 and omalizumab performed by Surface Plasmon Resonance (SPR).

FIG. 2 shows kinetic analyses of IgE binding to the anti-IgE monoclonal antibodies UB-221 vs. ligelizumab vs. omalizumab performed by Surface Plasmon Resonance (SPR).

FIG. 3 shows competitive neutralization by UB-221 vs. omalizumab of IgE binding to (upper panel), and inhibition of degranulation from basophilic FcεRI-expressing RBX SX-38 induced by IgE-specific ovalbumin (OVA) (lower panel).

FIG. 4 shows the reduction of serum humanized IgE in hIGHE-knockin mice treated with one single intravenous (IV) dose of UB-221 vs. omalizumab.

FIGS. 5A to 5C show ex vivo neutralization of high-level IgE in sera of atopic dermatitis patients by UB-221 vs. ligelizumab vs. omalizumab, including low IgE (<4,800 ng/mL, FIG. 5A), medium IgE (4,800-24,000 ng/mL, FIG. 5B), and high IgE (>24,000 ng/mL, FIG. 5C).

FIG. 6 shows the binding of UB-221 vs. ligelizumab vs. omalizumab in free form to CD23-bound IgE (upper panel) and the binding in mAb:IgE complex form to CD23 (lower panel).

FIGS. 7A and 7B show the effects on IgE neo-synthesis in human PBMCs by UB-221 vs. ligelizumab vs. omalizumab, including the data with doses at 1, 3 and 10 μg/mL (FIG. 7A) and the data with doses at 10, 20 and 80 μg/mL (FIG. 7B).

FIGS. 8A to 8D show that UB-221 can bind cynomolgus macaque's IgE and CD23-bound IgE and induce a rapid, profound serum IgE reduction after a single IV dose, including the data showing that UB-221 can bind to cIgE (FIG. 8A), the data showing that UB-221 can dose-dependently engage in cCD23-bound cIgE (FIG. 8B), the pharmacokinetics (PK) data of UB-221 after an IV 5 mg/kg dose (FIG. 8C), and the data showing that UB-221 could induce a rapid, profound reduction of serum free cIgE (FIG. 8D).

FIG. 9 shows the design of the phase 1 clinical trial to evaluate the safety, tolerability, pharmacokinetics (PK), and pharmacodynamics (PD) in 15 chronic spontaneous urticaria patients administered with a single IV dose of UB-221 at 0.2, 0.6, 2, 6, and 10 mg/kg.

FIG. 10 shows UB-221 concentrations that decayed with a dose-dependent half-live up to 22 days in sera of chronic spontaneous urticaria patients after treatment with a single IV dose of UB-221.

FIGS. 11A to 11C show that UB-221 at a single IV dose is efficacious in the treatment of chronic spontaneous urticaria as demonstrated by rapid reduction of UAS7 disease scores, individual (FIG. 11A) and mean (FIG. 11B), and daily changes in UAS disease score (FIG. 11C).

FIGS. 12A and 12B show that UB-221 at a single IV dose is efficacious in the treatment of chronic spontaneous urticaria as demonstrated by rapid reduction of HSS7 scores, individual (FIG. 12A) and mean (FIG. 12B).

FIG. 13 shows individual free IgE concentrations in sera of chronic spontaneous urticaria patients after treatment with a single IV dose of UB-221.

FIG. 14 shows concurrent mean serum UB-221, free IgE concentrations and UAS7 scores in chronic spontaneous urticaria patients receiving a single IV dose of UB-221.

FIG. 15 shows the amino acid sequences of the heavy chain variable region (V_{H}) and light chain variable region (V_{L}) of UB-221. Complementary determining regions (CDRs) are underlined, including GYTFNGYWMH (HC CDR1, SEQ ID NO: 2), YINPTTGHTEYNQKFKD (HC CDR2, SEQ ID NO: 4), ARQEYRHSWFAY (HC CDR3, SEQ ID NO: 6), QSVDYDGDTYM (LC CDR1, SEQ ID NO: 9), AASNLDS (LC CDR2, SEQ ID NO: 11), and QQTNEDPWT (LC CDR3, SEQ ID NO: 13).

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

### I. DEFINITIONS

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, 20 or 10 amino acid residues).

As used herein, the term “approximately” or “about” refers to a degree of acceptable deviation that will be understood by persons of ordinary skill in the art, which may vary to some extent depending on the context in which it is used. Specifically, “approximately” or “about” may mean a numeric value having a range of ±10% or ±5% or ±3% around the cited value.

As used herein, the term “substantially identical” refers to two sequences having 80% or more, preferably 85% or more, more preferably 90% or more, even more preferably 95% or more homology.

As used herein, the term “antibody” (interchangeably used in plural form, antibodies) means an immunoglobulin molecule having the ability to specifically bind to a particular target antigenic molecule. As used herein, the term “antibody” includes not only intact (i.e., full-length) antibody molecules but also antigen-binding fragments thereof retaining antigen-binding ability, e.g., Fab, Fab′, F(ab′)2 and Fv. Such fragments are also well known in the art and are regularly employed both in vitro and in vivo. The term “antibody” also includes chimeric antibodies, humanized antibodies, human antibodies, diabodies, linear antibodies, single chain antibodies, multispecific antibodies (e.g., bispecific antibodies) and any other modified configuration of the immunoglobulin molecule that comprises an antigen recognition site of the required specificity, including amino acid sequence variants of antibodies, glycosylation variants of antibodies, and covalently modified antibodies.

An intact or complete antibody comprises two heavy chains and two light chains. Each heavy chain contains a variable region (V_{H}) and first, second and third constant regions (C_{H}1, C_{H}2 and C_{H}3); and each light chain contains a variable region (V_{L}) and a constant region (C_{L}). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light chains and those of heavy chains are responsible for antigen binding. The variables regions in both chains are responsible for antigen binding generally, each of which contains three highly variable regions, called the complementarity determining regions (CDRs); namely, heavy (H) chain CDRs including HC CDR1, HC CDR2, HC CDR3 and light (L) chain CDRs including LC CDR1, LC CDR2, and LC CDR3. The three CDRs are franked by framework regions (FR1, FR2, FR3, and FR4), which are more highly conserved than the CDRs and form a scaffold to support the hypervariable regions. The constant regions of the heavy and light chains are not responsible for antigen binding, but are involved in various effector functions. Depending on the antibody amino acid sequence of the constant domain of its heavy chains, immunoglobulins can be assigned to different classes. There are five major classes of immunoglobulins: IgA, IgD, IgE, IgG, and IgM. The heavy-chain constant domains that correspond to the different classes of immunoglobulins are called alpha, delta, epsilon, gamma, and mu, respectively.

As used herein, the term “antigen-binding fragment” or “antigen-binding domain” refers to a portion or region of an intact antibody molecule that is responsible for antigen binding. An antigen-binding fragment is capable of binding to the same antigen to which the parent antibody binds. Examples of antigen-binding fragments include, but are not limited to: (i) a Fab fragment, which can be a monovalent fragment composed of a V_{H}-C_{H}1 chain and a V_{L}-C_{L} chain; (ii) a F(ab′)2 fragment which can be a bivalent fragment composed of two Fab fragments linked by a disulfide bridge at the hinge region; (iii) an Fv fragment, composed of the V_{H} and V_{L} domains of an antibody molecule associated together by noncovalent interaction; (iv) a single chain Fv (scFv), which can be a single polypeptide chain composed of a V_{H} domain and a V_{L} domain via a peptide linker; and (v) an (scFv)₂, which can contain two V_{H} domains linked by a peptide linker and two V_{L} domains, which are associated with the two V_{H} domains via disulfide bridges.

As used herein, the term “chimeric antibody” refers to an antibody containing polypeptides from different sources, e.g., different species. In some embodiments, in chimeric antibodies, the variable region of both light and heavy chains may mimic the variable region of antibodies derived from one species of mammal (e.g., a non-human mammal such as mouse, rabbit and rat), while the constant region may be homologous to the sequences in antibodies derived from another mammal such as a human.

As used herein, the term “humanized antibody” refers to an antibody comprising a framework region originated from a human antibody and one or more CDRs from a non-human (usually a mouse or rat) immunoglobulin.

As used herein, the term “human antibody” refers to an antibody in which essentially the entire sequences of the light chain and heavy chain sequences, including the complementary determining regions (CDRs), are from human genes. In some circumstances, the human antibodies may include one or more amino acid residues not encoded by human germline immunoglobulin sequences, e.g., by mutations in one or more of the CDRs, or in one or more of the FRs, such as to, for example, decrease possible immunogenicity, increase affinity, and eliminate cysteines that might cause undesirable folding.

As used herein, the term “specific binds” or “specifically binding” refers to a non-random binding reaction between two molecules, such as the binding of the antibody to an epitope of its target antigen. An antibody that “specifically binds” to a target antigen or an epitope is a term well understood in the art, and methods to determine such specific binding are also well known in the art. An antibody “specifically binds” to a target antigen if it binds with greater affinity/avidity, more readily, and/or greater duration than it binds to other substances. In other words, it is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, the affinity of the binding can be defined in terms of a dissociation constant (K_{D}). Typically, specifically binding when used with respect to an antibody can refer to an antibody that specifically binds to (recognize) its target with a K_{D} value less than about 10⁻⁸ M, such as about 10⁻⁹ M or less, about 10⁻¹⁰ M or less, about 10⁻¹¹ M or less, about 10⁻¹² M or less, or even less, and binds to the specific target with an affinity corresponding to a K_{D} that is at least ten-fold lower than its affinity for binding to a non-specific antigen (such as BSA or casein), such as at least 100 fold lower, e.g., at least 1,000 fold lower or at least 10,000 fold lower.

As used herein, the term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single-stranded or double-stranded form.

As used herein, the term “complementary” refers to the topological compatibility or matching together of interacting surfaces of two polynucleotides. A first polynucleotide is complementary to a second polynucleotide when the nucleotide sequence of the first polynucleotide is identical to the nucleotide sequence of the polynucleotide binding partner of the second polynucleotide. Thus, the polynucleotide whose sequence 5′-ATATC-3′ is complementary to a polynucleotide whose sequence is 5′-GATAT-3′.”

As used herein, the term “encoding” refers to the natural property of specific sequences of nucleotides in a polynucleotide (e.g., a gene, a cDNA, or an mRNA) to serve as templates for synthesis of other polymers and macromolecules in biological processes having either a given sequence of RNA transcripts (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

As used herein, the term “recombinant nucleic acid” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it or transferring it between different locations (e.g., between different organisms). Vectors can be introduced into a suitable host cell for the above-described purposes. A “recombinant cell” refers to a host cell that has introduced a recombinant nucleic acid into it. “A transformed cell” mean a cell into which has been introduced, by means of recombinant DNA techniques, a DNA molecule encoding a protein of interest.

Vectors may be of various types, including plasmids, cosmids, episomes, fosmids, artificial chromosomes, phages, viral vectors, etc. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example, and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, a secretion signal sequence (e.g., α-mating factor signal), a stop codon, and other control sequences (e.g., Shine-Dalgarno sequences and termination sequences). Preferably, vectors may further contain a marker sequence (e.g., an antibiotic-resistant marker sequence) for the subsequent screening/selection procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes a tag for purpose of purification, e.g., a His-tag.

As used herein, the term “treatment” refers to the application or administration of one or more active agents to a subject afflicted with a disorder, a symptom or condition of the disorder, or a progression of the disorder, with the purpose to cure, heal, alleviate, relieve, alter, remedy, ameliorate, improve, or affect the disorder, the symptom or condition of the disorder, the disabilities induced by the disorder, or the progression or predisposition of the disorder.

### II. ANTIBODIES DIRECTED TO IGE

According to the present invention, an anti-IgE antibody, as used herein, is a multifunctional anti-IgE antibody which neutralizes IgE and inhibits IgE synthesis.

As described herein, a neutralizing anti-IgE antibody may partially or fully inhibit the binding of IgE to FcεRI on mast cells and basophil cells and may cause any substantial inhibition or blocking of IgE signaling through FcεRI that is responsible for allergic hypersensitivity and inflammation. Neutralization can be measured by any suitable method. In one aspect, IgE neutralization is reflected in that the neutralizing antibody can cause at least about 30%, preferably at least about 40%, at least about 50%, at least about 60%, at least about 75% or more (e.g., about 25-100%) inhibition in degranulation induced by IgE-mediated antigen stimulation compared to the amount of degranulation that typically occurs in a substantially identical condition without the presence of the neutralizing antibody.

As described herein, IgE synthesis includes de novo IgE production. An IgE level and inhibition of IgE synthesis can be measured by any suitable method. In one aspect, an inhibitory antibody that substantially inhibits IgE synthesis can cause 35%, 40%, 45%, 50%, 55%, 60%, 65%, 70%, 75%, 80%, 85%, 90%, 95% or more decrease in an IgE level from the cells treated with the inhibitory antibody, compared to an IgE level from the cells under a substantially identical condition without the presence of the inhibitory antibody. Specifically, an inhibitory antibody that inhibits IgE synthesis involves interaction with CD23 in free form or IgE-complex form or both. More specifically, an inhibitory antibody inhibits IgE synthesis via interaction with CD23 in both free form and IgE-complex form and thus substantially inhibits or blocks CD23-mediated downregulation of IgE production.

In some embodiments, an anti-IgE antibody, as described herein, is an anti-IgE monoclonal antibody, UB-221 mAb. The amino acid sequences of the heavy chain variable region (V_{H}) and light chain variable region (V_{L}), and their complementary determining regions (HC CDR1, HC CDR2 and HC CDR3) (LC CDR1, LC CDR2 and LC CDR3) of UB-221 mAb are as shown in Table 1 below. The anti-IgE antibody of the present invention includes UB-221 mAb and its functional variant.

V_{H} domain

| FR1 | CDR1 | | FR2 | CDR2 |
|---|---|---|---|---|
| QVQLVQSGAEVKKPGSSV | GYTFNGYWMH | | WVRQAPGQGLEWIG | YINPTTGHTEYN |
| KVSCKAS | (SEQ ID NO: 2) | | (SEQ ID NO: 3) | QKFKD |
| (SEQ ID NO: 1) | | | | (SEQ ID NO: 4) |
| FR3 | | CDR3 | FR4 | |

| | | |
|---|---|---|
| KATITADESTNTAYMELSS | ARQEYRHSWF | WGQGTLVTVSS |
| LRSEDTAVYYC | AY | (SEQ ID NO: 7) |
| (SEQ ID NO: 5) | (SEQ ID NO: 6) | |

V_{L} domain

| FR1 | CDR1 | FR2 | CDR2 |
|---|---|---|---|
| DIQLTQSPSSLSASVGDRV | QSVDYDGDTYM | NWYQQKPGKAPKLLIY | AASNLDS |
| TITCRAS | (SEQ ID NO: 9) | (SEQ ID NO: 10) | (SEQ ID NO: 11) |
| (SEQ ID NO: 8) | | | |
| FR3 | CDR3 | FR4 | |
| GVPSRFSGSGSGIDFTLTISSLQP | QQTNEDPWT | FGQGTKVEIKR | |
| EDFATYYC | (SEQ ID NO: 13) | (SEQ ID NO: 14) | |
| (SEQ ID NO: 12) | | | |

Full-length amino acid sequences of heavy chain and light chain

| | |
|---|---|
| heavy chain | QVQLVQSGAEVKKPGSSVKVSCKASGYTFNGYWMHWVRQAPGQGLEWIGYINP |
| | TTGHTEYNQKFKDKATITADESTNTAYMELSSLRSEDTAVYYCARQEYRHSWFAY |
| | WGQGTLVTVSS |
| | (SEQ ID NO: 15) |
| light chain | DIQLTQSPSSLSASVGDRVTITCRASQSVDYDGDTYMNWYQQKPGKAPKLLIYAAS |
| | NLDSGVPSRFSGSGSGTDFTLTISSLQPEDFATYYCQQTNEDPWTFGQGTKVEIKR |
| | (SEQ ID NO: 16) |

In some embodiments, the anti-IgE antibody of the present invention is a functional variant of UB-221 mAb which is characterized in comprising (a) a VH comprising HC CDR1 of SEQ ID NO: 2, HC CDR2 of SEQ ID NO: 4, and HC CDR3 of SEQ ID NO: 6; and (b) a V_{L} comprising LC CDR1 of SEQ ID NO: 9, LC CDR2 of SEQ ID NO: 11, and HC CDR3 of SEQ ID NO: 13, or an antigen-binding fragment thereof.

In some embodiments, the anti-IgE antibody of the present invention, having (a) a VH comprising HC CDR1 of SEQ ID NO: 2, HC CDR2 of SEQ ID NO: 4, and HC CDR3 of SEQ ID NO: 6; and (b) a V_{L} comprising LC CDR1 of SEQ ID NO: 9, LC CDR2 of SEQ ID NO: 11, and LC CDR3 of SEQ ID NO: 13, can comprise a V_{H} comprising SEQ ID NO: 15 or an amino acid sequence substantially identical thereto and a V_{L} comprising SEQ ID NO: 16 or an amino acid sequence substantially identical thereto. Specifically, the anti-IgE antibody of the present invention includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO:15, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO:16. The anti-IgE antibody of the present invention also includes any recombinantly (engineered)-derived antibody encoded by the polynucleotide sequence encoding the relevant V_{H} or V_{L} amino acid sequences as described herein.

U.S. Pat. No. 10,047,166 describes one example of an anti-IgE antibody of the present invention, the relevant disclosures of each of which are incorporated by reference herein for the purposes or subject matter referenced herein.

The term “substantially identical” can mean that the relevant amino acid sequences (e.g., in FRs, CDRs, V_{H}, or V_{L}) of a variant differ insubstantially as compared with a reference antibody such that the variant has substantially similar binding activities (e.g., affinity, specificity, or both) and bioactivities relative to the reference antibody. Such a variant may include minor amino acid changes. It is understandable that a polypeptide may have a limited number of changes or modifications that may be made within a certain portion of the polypeptide irrelevant to its activity or function and still result in a variant with an acceptable level of equivalent or similar biological activity or function. In some examples, the amino acid residue changes are conservative amino acid substitution, which refers to the amino acid residue of a similar chemical structure to another amino acid residue and the polypeptide function, activity or other biological effect on the properties smaller or substantially no effect. Typically, relatively more substitutions can be made in FR regions, in contrast to CDR regions, as long as they do not adversely impact the binding function and bioactivities of the antibody (such as reducing the binding affinity by more than 50% as compared to the original antibody). In some embodiments, the sequence identity can be about 80%, 82%, 84%, 85%, 86%, 88%, 90%, 92%, 94%, 95%, 96%, 98%, or 99%, or higher, between the reference antibody and the variant. Variants can be prepared according to methods for altering polypeptide sequence known to one of ordinary skills in the art such as those found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989. For example, conservative substitutions of amino acids include substitutions made amongst amino acids within the following groups: (i) A, G; (ii) S, T; (iii) Q, N; (iv) E, D; (v) M, I, L, V; (vi) F, Y, W; and (vii) K, R, H.

The antibodies described herein may be animal antibodies (e.g., mouse-derived antibodies), chimeric antibodies (e.g., mouse-human chimeric antibodies), humanized antibodies, or human antibodies. The antibodies described herein may also include their antigen-binding fragments e.g., a Fab fragment, a F(ab′)2 fragment, an Fv fragment, a single chain Fv (scFv) and an (scFv)₂. The antibodies or their antigen-binding fragments can be prepared by methods known in the art.

### III. PREPARATION OF ANTIBODIES

Numerous methods conventional in this art are available for obtaining antibodies or antigen-binding fragments thereof.

In some embodiments, the antibodies provided herein may be made by the conventional hybridoma technology. In general, a target antigen optionally coupled to a carrier protein and/or mixed with an adjuvant may be used to immunize a host animal for generating antibodies binding to that antigen. Lymphocytes secreting monoclonal antibodies are harvested and fused with myeloma cells to produce hybridoma. Hybridoma clones formed in this manner are then screened to identify and select those that secrete the desired monoclonal antibodies.

In some embodiments, the antibodies provided herein may be prepared via recombinant technology. In related aspects, isolated nucleic acids that encode the disclosed amino acid sequences, together with vectors comprising such nucleic acids and host cells transformed or transfected with the nucleic acids, are also provided.

For example, nucleic acids comprising nucleotide sequences encoding the heavy and light chain variable regions of such an antibody can be cloned into expression vectors (e.g., a bacterial vector such as an E. coli vector, a yeast vector, a viral vector, or a mammalian vector) via routine technology, and any of the vectors can be introduced into suitable cells (e.g., bacterial cells, yeast cells, plant cells, or mammalian cells) for expression of the antibodies. Examples of mammalian host cell lines are human embryonic kidney line (293 cells), baby hamster kidney cells (BHK cells), Chinese hamster ovary cells (CHO cells), African green monkey kidney cells (VERO cells), and human liver cells (Hep G2 cells). The recombinant vectors for expression of the antibodies described herein typically contain a nucleic acid encoding the antibody amino acid sequences operably linked to a promoter, either constitutive or inducible. Typical vectors contain transcription and translation terminators, initiation sequences, and promoters useful for the regulation of the expression of the nucleic acid encoding the antibody. The vectors optionally contain selection markers for both prokaryotic and eukaryotic systems. In some examples, both the heavy and light chain coding sequences are included in the same expression vector. In other examples, each of the heavy and light chains of the antibody is cloned into an individual vector and produced separately, which can be then incubated under suitable conditions for antibody assembly.

The recombinant vectors for expression the antibodies described herein typically contain a nucleic acid encoding the antibody amino acid sequences operably linked to a promoter, either constitutive or inducible. The recombinant antibodies can be produced in prokaryotic or eukaryotic expression systems, such as bacteria, yeast, insect and mammalian cells. Typical vectors contain transcription and translation terminators, initiation sequences, and promoters useful for regulation of the expression of the nucleic acid encoding the antibody. The vectors optionally contain selection markers for both prokaryotic and eukaryotic systems. The antibody protein as produced can be further isolated or purified to obtain preparations that are substantially homogeneous for further assays and applications. Suitable purification procedures, for example, may include fractionation on immunoaffinity or ion-exchange columns, ethanol precipitation, sodium dodecyl sulfate polyacrylamide gel electrophoresis (SDS-PAGE), high-performance liquid chromatography (HPLC), ammonium sulfate precipitation, and gel filtration.

When a full-length antibody is desired, coding sequences of any of the V_{H} and V_{L} chains described herein can be linked to the coding sequences of the Fc region of an immunoglobulin and the resultant gene encoding a full-length antibody heavy and light chains can be expressed and assembled in a suitable host cell, e.g., a plant cell, a mammalian cell, a yeast cell, or an insect cell.

Antigen-binding fragments can be prepared via routine methods. For example, F(ab′)₂ fragments can be generated by pepsin digestion of a full-length antibody molecule, and Fab fragments that can be made by reducing the disulfide bridges of F(ab′)₂ fragments. Alternatively, such fragments can also be prepared via recombinant technology by expressing the heavy and light chain fragments in suitable host cells and have them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

### IV. COMPOSITIONS

According to the present invention, the anti-IgE antibody may be formulated with a pharmaceutically acceptable carrier into a composition for purpose of delivery and absorption.

As used herein, “pharmaceutically acceptable” means that the carrier is compatible with an active ingredient in the composition, and preferably can stabilize said active ingredient and is safe to the receiving individual. Said carrier may be a diluent, vehicle, excipient, or matrix to the active ingredient. Typically, a composition comprising an anti-IgE antibody as described herein as an active ingredient can be in a form of a solution such as an aqueous solution i.e., a saline solution or it can be provided in powder form. The composition may further contain pharmaceutically acceptable auxiliary substances as required to approximate physiological conditions, for example, pH adjusting and buffering agents, such as sodium acetate, sodium chloride, potassium chloride, calcium chloride, sodium lactate and the like. The form of the composition may be suspensions, lotions, solutions, sterilized injection fluid, and packaged powder. The composition of the present invention may be delivered via any physiologically acceptable route, such as parenteral (such as intramuscular, intravenous, subcutaneous, and intraperitoneal) and intranasal methods. In certain embodiments, the composition of the present invention is administered as a liquid injectable formulation which can be provided as a ready-to-use dosage form or as a reconstitutable stable powder.

### V. TREATMENT

The present invention provides a method for treating an IgE-mediated disease by administering a multifunctional anti-IgE antibody as described herein. The method of the present invention is effective in providing rapid and/or sustained symptomatic relief, and a less frequent dosing is required that increases patient comfort and convenience.

As used herein, the term “symptomatic relief” may mean an active agent that reduces or eliminates one or more perceived symptoms of a disease or other abnormal state. The symptomatic severity level of a disease may be determined by any suitable index or score known in the art. In general, a higher level of the index or a higher score indicates greater disease severity. In some embodiments, symptomatic relief may include a reduced level of such index or a decreased score, for example, by 5%, 10%, 15%, 20%, 25%, 30%, 45%, 50%, 55%, 60%, 65%, 70%, 75%, 80%, 85%, 90%, 95% or more, compared with a diseased level of the index or a diseased score that one of ordinary skill in the art and/or a medical professional, e.g., a doctor would expect a diseased individual or population of similar physical characteristics and medical history to have. In some embodiments, symptomatic relief may mean that one or more perceived symptoms of a disease or other abnormal states are alleviated to a normal status.

In some embodiments, the symptomatic relief as described herein may include IgE reduction, alleviation in itch and/or decrease of hive counts for CSU. Specifically, two score systems have been used to score the signs and symptoms of CSU, including: 1) UAS7 (Urticaria Activity Score) based scoring daily the number of daily wheals and itching intensity on a scale of 0 to 3 over 7 days; and 2) HSS7 (Hives Severity Score) based on scoring daily the number of hives on a scale of 0 to 3 over 7 days. UAS7 scores are grouped in 5 score bands: (i) urticaria-free, score 0, (ii) well-controlled urticarial, score 1-6; (iii) mild urticarial, score 7-15; (iv) moderate urticarial, score 16-27; and (v) severe urticarial, score 28-42. HSS7 scores are grouped in 4 score bands: (i) none, score 0; (ii) mild (1-6 hives/12 hours); (iii) moderate (7-12 hives/12 hours); and (iv) severe (>12 hives/12 hours). In certain examples, the symptomatic relief as described herein may mean that the condition of a patient is improved from a severe status to a moderate, mild or symptom-free status.

As used herein, the term “rapid relief” may mean that relief from symptoms of a disease occurs within one week or faster (e.g., within 7 days, 6 days, 5 days, 4 days, 3 days, 2 days or 1 day, i.e., 24 hours) following administration of an active agent. With respect to “sustained relief” it refers to relief from symptoms of a disease that lasts in duration for 2 weeks or longer (e.g., 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks) after administration of an active agent. Specifically, the term “rapid and sustained relief” may mean that immediate relief starts within one week or faster (e.g., within 7 days, 6 days, 5 days, 4 days, 3 days, 2 days or 1 day i.e., 24 hours or less) following administration of an active agent and continues and remains constant and stable for at least 2 weeks or longer (e.g., 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks or longer), particularly at least 3 weeks or longer (e.g., 3 to 14 weeks or longer), at least 4 weeks or longer (e.g., 4 to 14 weeks or longer), at least 5 weeks or longer (e.g., 5 to 14 weeks or longer), at least 6 weeks or longer (e.g., 6 to 14 weeks or longer), at least 7 weeks or longer (e.g., 7 to 14 weeks or longer), at least 8 weeks or longer (e.g., 8 to 14 weeks or longer), at least 9 weeks or longer (e.g., 9 to 14 weeks or longer), at least 10 weeks or longer (e.g., 10 to 14 weeks or longer), at least 11 weeks or longer (e.g., 11 to 14 weeks or longer), at least 12 weeks or longer (e.g., 12 to 14 weeks or longer), at least 13 weeks or longer (e.g., 13 to 14 weeks or longer), or at least 14 weeks or longer. In some embodiments, the relief may continue at least 8 to 24 weeks (2 to 6 months), at least 12 to 24 weeks (3 to 6 months), at least 16 to 24 weeks (4 to 6 months), at least 20 to 24 weeks (5 to 6 months) or at least 24 weeks (6 months).

As used herein, the term “less frequent” or “less frequently” with respect to dosing may mean that an active agent (e.g., UB-221) for treating a disease is administered every 2 to 14 weeks or less frequently, for example, every 3 weeks, every 4 weeks, every 5 weeks, every 6 weeks, every 7 weeks, every 8 weeks, every 9 weeks, every 10 weeks, every 11 weeks, every 12 weeks, every 13 weeks, or every 14 weeks or less frequently. In some embodiments, the active agent (e.g., UB-221) is administered every 3 to 14 weeks or less frequently, every 4 to 14 weeks or less frequently, every 5 to 14 weeks or less frequently, every 6 to 14 weeks or less frequently, every 7 to 14 weeks or less frequently, every 8 to 14 weeks or less frequently, every 9 to 14 weeks or less frequently, every 10 to 14 weeks or less frequently, every 11 to 14 weeks or less frequently, every 12 to 14 weeks or less frequently, every 13 to 14 weeks or less frequently, or every 14 weeks or less frequently. In some embodiments, the active agent (e.g., UB-221) is administered every 8 to 24 weeks (every 2 to 6 months), every 12 to 24 weeks (every 3 to 6 months), every 16 to 24 weeks (every 4 to 6 months), every 20 to 24 weeks (every 5 to 6 months) or every 24 weeks (every 6 months).

In some embodiments, a multifunctional anti-IgE antibody as described herein as an active agent for treating an IgE-mediated disease provides sustained symptomatic relief which may last longer in duration and requires less frequent dosing than when the subject receives other treatments, for example, small molecule drugs, cyclic peptide drugs, or other biologics. Small molecule drugs (e.g., anti-histamine and cyclic peptide drugs [e.g., cyclosporine]) are usually administered with high dosing frequencies, such as daily medication with pills or capsules for long weeks. Examples of other biologics e.g., omalizumab and ligelizumab (anti-IgE antibodies) are administered every 2 or 4 weeks. Such anti-IgE antibodies are deemed as single functional anti-IgE antibodies (or called a plain IgE neutralizer), which are capable of neutralizing IgE but fail to provide a substantial inhibition of de novo IgE production (e.g., causing 35%, 40%, 45%, 50%, 55%, 60%, 65%, 70%, 75%, 80%, 85%, 90%, 95% or more decrease in the IgE level). Specifically, such single functional anti-IgE antibodies react limited or stay inert toward CD23 and thus do not substantially inhibit CD23-mediated downregulation of IgE production.

The method of the present invention is effective in treating an IgE-mediated disease, including allergic or non-allergic. Examples of an IgE-mediated disease as described herein include but are not limited to allergic asthma, allergic rhinitis, atopic dermatitis, food allergy, chronic spontaneous (idiopathic) urticaria, chronic rhinosinusitis, systemic mastocytosis, cutaneous mastocytosis, allergic bronchopulmonary aspergillosis, recurrent idiopathic angioedema, or eosinophil-associated gastrointestinal disorder.

The term “effective amount” used herein refers to the amount of an active ingredient to confer a desired biological effect in a treated subject or cell. For example, an effective amount, as described herein, may be an amount of a multifunctional anti-IgE antibody as an active agent that can provide substantial inhibition of de novo IgE production and lead to a rapid and/or sustained symptomatic relief for an IgE-mediated disease. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience.

In some embodiments, a multifunctional anti-IgE antibody as described herein is administered in a dose range from 0.1 to 10 mg, preferably 0.3 to 10 mg, more preferably 0.5 to 10 mg, still more preferably 1 to 10 mg, and even more preferably 2 to 10 mg, 3 to 10 mg, 4 to 10 mg, 5 to 10 mg, 6 to 10 mg, 7 to 10 mg, 8 to 10 mg, or 10 mg per kilogram of body weight of the subject.

In some embodiments, the antibody is included in a composition in an entire dose and is administered to the subject in a single dose.

A subject to be treated by the method of treatment as described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder. A subject suspected of having any of such target disease/disorder might show one or more symptoms of the disease/disorder. A subject at risk for the disease/disorder can be a subject having one or more of the risk factors for that disease/disorder.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### Example 1: Determination of IgE Binding Affinity of UB-221 vs. Omalizumab on Surface Plasmon Resonance (SPR) Analysis

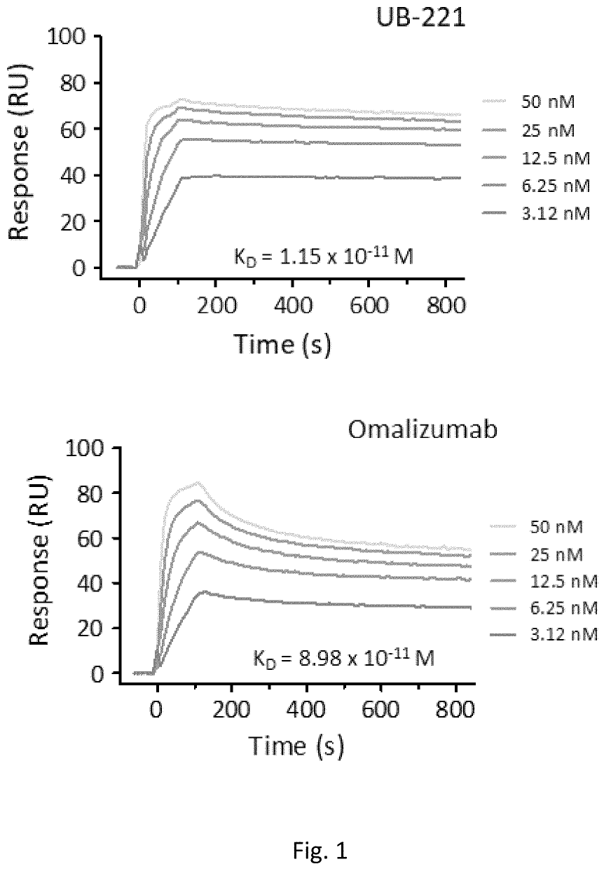

Kinetic analyses of IgE binding to the anti-IgE monoclonal antibodies UB-221 (UBP product from parent CHO-S clone) and omalizumab (Novartis, Xolair® CA-075) were performed by Surface Plasmon Resonance (SPR) technology using Biacore X100 instrument (GE Healthcare). Anti-human IgG Fc fragment antibodies (BR-1008-39, GE) were first immobilized by amino coupling to the surface of CMS gold sensor chip, to which the diluted anti-IgE antibodies at 1 μg/mL were captured. The Expi293-expressed human full-length IgE samples at 0.312-50 nM diluted in HBS-EP buffer were injected for 120 seconds and the dissociation was measured for 720 seconds under constant buffer flow. The dissociation rate constant (Kd), association rate constant (Ka), and the equilibrium dissociation rate constant (K_{D}) were calculated using a 1:1 Langmuir curve fitting model. The results indicate that UB-221 binds to IgE with an affinity (K_{D}, 1.15×10⁻¹¹ M) ˜8-fold higher than by omalizumab (K_{D}, 8.98×10⁻¹¹M). The kinetic sensorgrams and binding affinities values are shown in FIG. 1.

### Example 2: Determination of IgE Binding Affinity of UB-221 vs. Ligelizumab Vs. Omalizumab on Surface Plasmon Resonance (SPR) Analysis

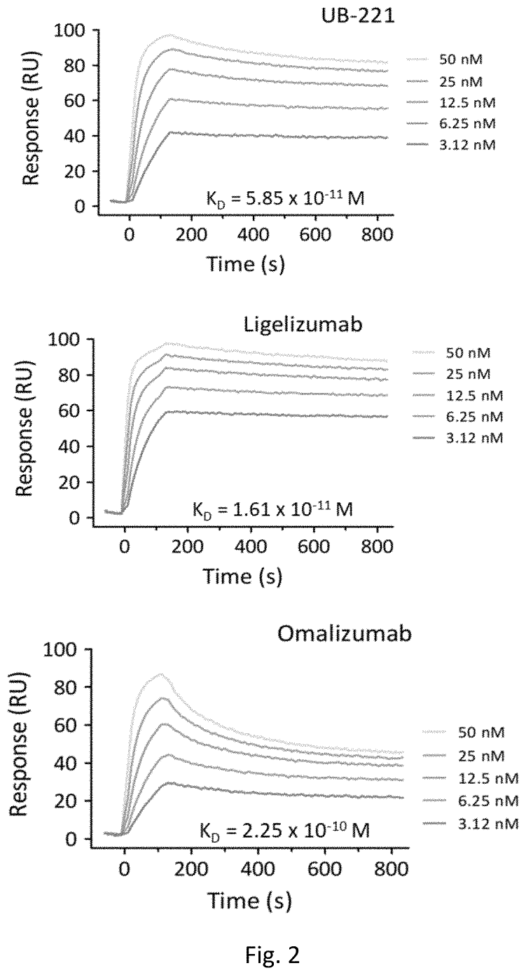

Kinetic analyses of IgE binding to the anti-IgE monoclonal antibodies UB-221 (UBP product from stable CHO-S clone), a surrogate antibody (SA) for ligelizumab (Creative Biolabs, CB190513) and omalizumab (Novartis, Xolair® DCA-075) were performed by Surface Plasmon Resonance (SPR) technology using Biacore X100 instrument (GE Healthcare). Anti-human IgG Fc fragment antibodies (BR-1008-39, GE) were first immobilized by amino coupling to the surface of CMS gold sensor chip, to which the diluted anti-IgE antibodies at 1 μg/mL were captured. The Expi293-expressed human full-length IgE samples at 0.312-50 nM diluted in HBS-EP buffer were injected for 120 seconds and the dissociation was measured for 720 seconds under constant buffer flow. The dissociation rate constant (Kd), association rate constant (Ka), and the equilibrium dissociation rate constant (K_{D}) were calculated using a 1:1 Langmuir curve fitting model. The results indicate that ligelizumab binds to IgE with the greatest affinity (K_{D}, 1.61×10⁻¹¹ M), which is about 4-fold higher than by UB-221 (K_{D}, 5.85×10⁻¹¹ M), and about 14-fold higher than by omalizumab. The kinetic sensorgrams and binding affinities values are shown in FIG. 2.

### Example 3: Competitive Inhibitions on IgE:FcεRI Interaction and Basophil Degranulation

3.1 Inhibition of IgE Binding to FIERI-Expressing RBL SX-38 Cells

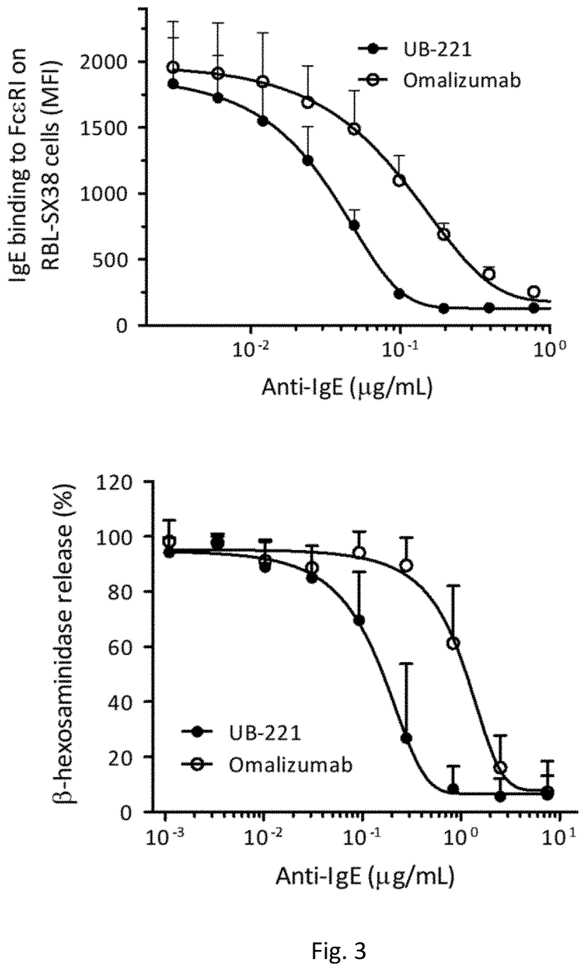

RBL SX-38 cells at 1×10⁵ were mixed with 400 ng/mL of recombinant human IgE for 1 hour on ice. After washing three times with 1% BSA/PBS, UB-221 and omalizumab at their concentrations ranging from 25,000 to 6 ng/mL were added into cells and incubated for 1 hour on ice. Finally, the cells were washed three times with 1% BSA/PBS and incubated with 0.75 μg/mL of PE-conjugated anti-human IgE (eBioscience, CN. 12-6986, Lot. E11877-1636) for 30 min on ice. The stained cells were analyzed on a FACS Verse cytometer after three washes. In competitive inhibition of IgE binding to FcεRI-expressing RBL SX-38 (mean±SD, n=3) as shown in FIG. 3 (upper panel), UB-221 inhibits IgE biding with a 3-fold superiority over omalizumab (IC₅₀: 0.035 vs. 0.106 mg/mL).

3.2 RBL SX-38 Degranulation Assay

The β-hexosaminidase, a lysosomal enzyme released after stimulation, correlates well with the release of histamine and can thus be used to measure degranulation. RBL SX-38 cells (rat basophil cells expressing human FcεRI) were co-incubated with 1 μg/mL of OVA-specific hIgE (AllerMAbs, CN: OVA8G9-02) and 0.002 to 15 μg/mL of anti-IgE antibodies (UB-221 or omalizumab) for 2 hours at 37° C. The cells were then washed twice with culture medium, and degranulation reactions were induced with OVA/Triton X-100 (10 μg/ml OVA in 1% Triton X-100) for 30 min. β-hexosaminidase in cell supernatant was harvested and reacted by adding citric acid containing 4-MUG (4-methylumbelliferyl-N-acetyl-b-D-glucosaminide, Sigma-Aldrich, CN: M2133) for 1 hour. The resulting fluorescence (excitation 355 nm; emission 460 nm) was measured by the fluorescence reader. The percentages of degranulation expressed as % β-hexosaminidase release. In inhibition of RBL SX-38 degranulation induced by the IgE-specific ovalbumin (OVA)-IgE complex (mean±SD, n=6) as shown in FIG. 3 (lower panel), UB-221 reveals a 7-fold inhibitory superiority over omalizumab (IC₅₀: 0.14 vs. 0.94 mg/mL).

### Example 4: Reduction of Serum Humanized IgE in hIGHE-Knockin Mice Treated with a Single IV Dose of UB-221 vs. Omalizumab

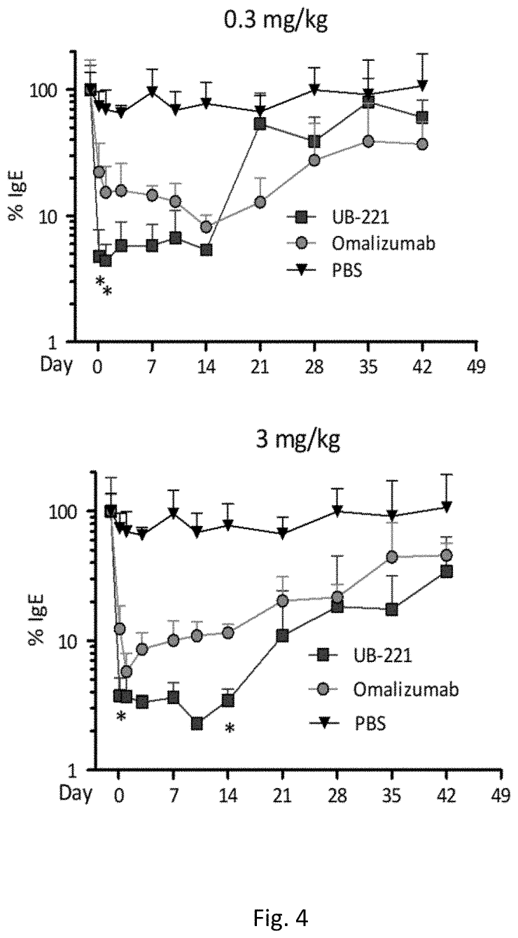

In human IgE knock-in C57BL/6 mice, in whose genome the Cg1 and Ck constant regions are replaced by human Ce and Ck constant regions, their IgE-secreting B cells can produce humanized IgE at a much higher level than mouse IgE. In the hIGHE-knockin mice (n=6) receiving a single i.p. dose of UB-221 or omalizumab at 0.3 or 3.0 mg/kg, UB-221 was observed to induce a rapid >90% reduction of serum free IgE at the low 0.3 mg/kg dose as shown in FIG. 4, while omalizumab would need a 10-fold higher dose (3.0 mg/kg) to achieve that level of IgE reduction. Data are shown as Mean±SEM. *P<0.05 indicates statistically significant.

### Example 5: Ex Vivo Neutralization of High-Level IgE in Sera of Atopic Dermatitis Patients

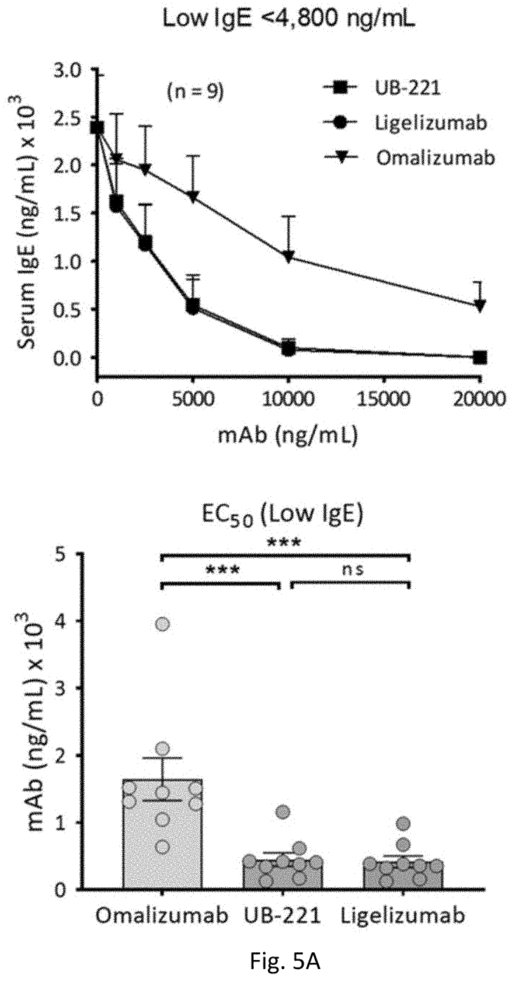

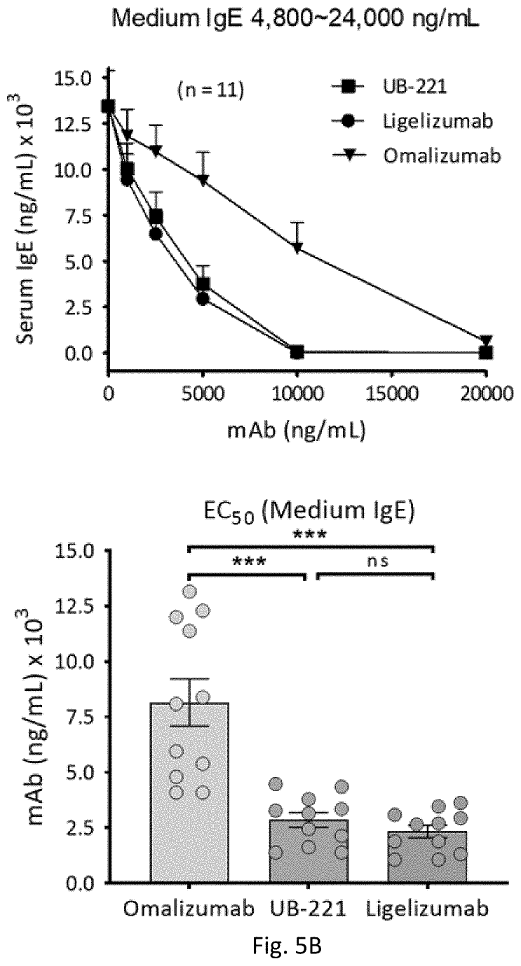

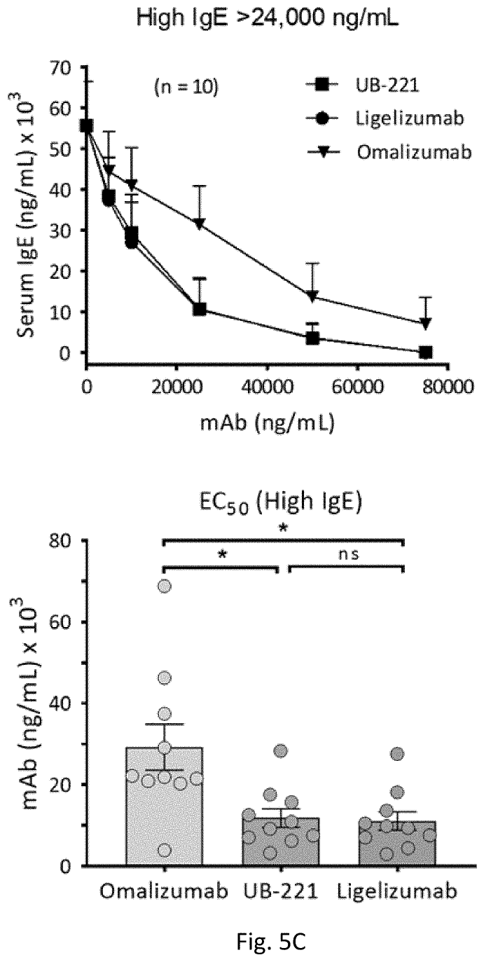

The potency in reduction of high IgE in sera from 30 patients with atopic dermatitis was compared for UB-221, ligelizumab and omalizumab, based on a competitive inhibition of IgE binding to the FcεRI immobilized on ELISA. The serum samples collected were grouped into three IgE ranges of low (<4,800 ng/mL, n=9), medium (4,800-24,000 ng/mL, n=11), and high IgE (>24,000 ng/mL, n=10). The serum samples were incubated with three escalating concentrations of anti-IgE mAbs. The comparisons were estimated by the Mann-Whitney U-test (P<0.001, ns=not significant). The results indicate that UB-221 and ligelizumab are equipotent in neutralizing serum IgE, while omalizumab is less potent as shown in FIGS. 5A to 5C, where, using the low IgE group as an example (FIG. 5A), the mAb drug concentrations to achieve a 50% IgE reduction (EC₅₀, Mean±SEM) were estimated to be 450±100, 419±100, and 1,647±317 ng/mL for UB-221, ligelizumab, and omalizumab, respectively.

### Example 6: Interactions with CD23 by Anti-IgE mAbs in Free or IgE-Complex Form

The 96-well ELISA plates were coated with 100 μL of 5 mg/mL CD23 and blocked with PBS-0.5% BSA. For assay of mAb binding to CD23-bound IgE, 100 μL of 100 ng/mL IgE in PBS-0.5% BSA was added and incubated for 1 hour at RT. After washing, serially diluted UB-221, omalizumab, and ligelizumab at 0.0001-100 μg/mL were added and incubated at RT for 1 hour. The binding of the anti-IgE mAbs was detected with Goat anti-human IgG Fc-HRP (Jackson Immuno Research, Inc. Cat. 109-035-098). Binding of anti-IgE mAbs in pre-formed IgE-complex to the CD23 was investigated using the same ELISA, with one modification that UB-221, omalizumab, and ligelizumab at 1.0-200 ng/mL were incubated at RT for 1 hour to allow the formation of complex, and the mixtures were added to the CD23-immobilized ELISA plates, then the anti-IgE mAbs bound on the CD23 was detected with Goat anti-human IgG Fc-HRP.

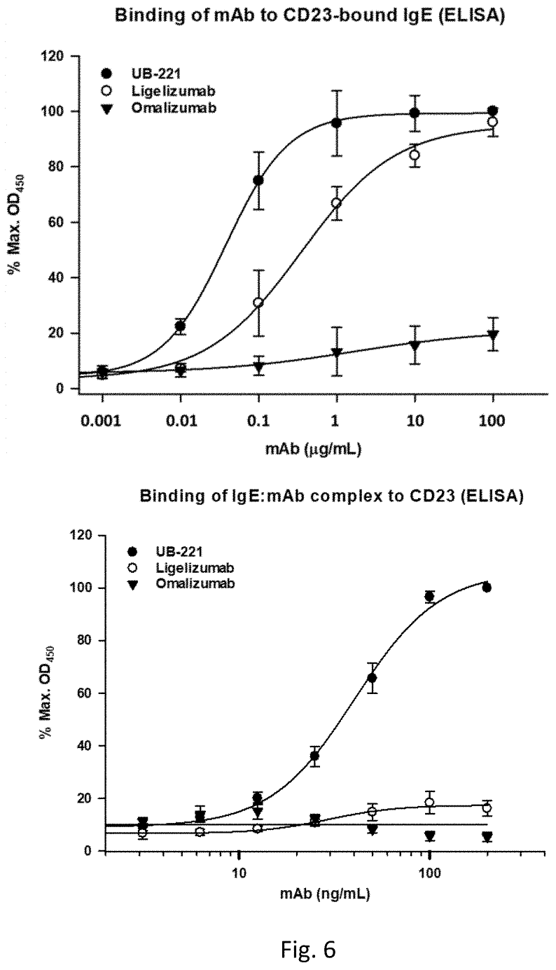

On CD23-immobilized ELISA with IgE preloaded, UB-221 in a free form exhibited a strong concentration-dependent binding to the CD23-bound IgE as shown in FIG. 6 (upper panel), estimated to be 10-fold more abundant than by ligelizumab as shown in EC₅₀ values (Mean±SD) of 38.4±3.6 vs. 402±47.3 ng/mL, while omalizumab was inactive. The pre-formed UB-221:IgE complex also exhibits a strong binding to CD23 as shown in FIG. 6 (lower panel) with an EC₅₀ of 41.6±4.6 ng/mL, nearly the same as that binding to the CD23-bound IgE, while ligelizumab in complex with IgE completely loses the ability to bind CD23, and the omalizumab:IgE complex stays inert toward CD23.

### Example 7: Reduction of IgE Neo-Synthesis in Human PBMCs by UB-221, Ligelizumab and Omalizumab

In the presence of UB-221, omalizumab, or ligelizumab, human PBMCs from healthy donors were stimulated with human recombinant IL-4 and an anti-human CD40 antibody to undergo de novo IgE synthesis. In one study with PBMCs from 14 blood donors (n=14) the effect on IgE production on Days 7 and 11 was investigated for UB-221 in comparison with omalizumab at drug doses of (a) 1 μg/mL, (b) 3 μg/mL, and (c) 10 μg/mL. In another study with 3-5 blood donors, the effect on IgE production focused on Day 11 by UB-221, omalizumab and ligelizumab at drug doses of (d) 10 μg/mL (n=3), (e) 20 μg/mL (n=5), and (f) 80 μg/mL (n=5). The total IgE in cell culture supernatant samples were quantified by ELISA. The percentages of IgE reduction were calculated with IgE levels from the respective untreated cells set as 100%. Data are shown as Mean±SEM. Different treatments were compared relative to the untreated group using two-way ANOVA with Tukey's multiple comparison referenced to untreated controls. *P<0.05, **P<0.01, ***P<0.001.

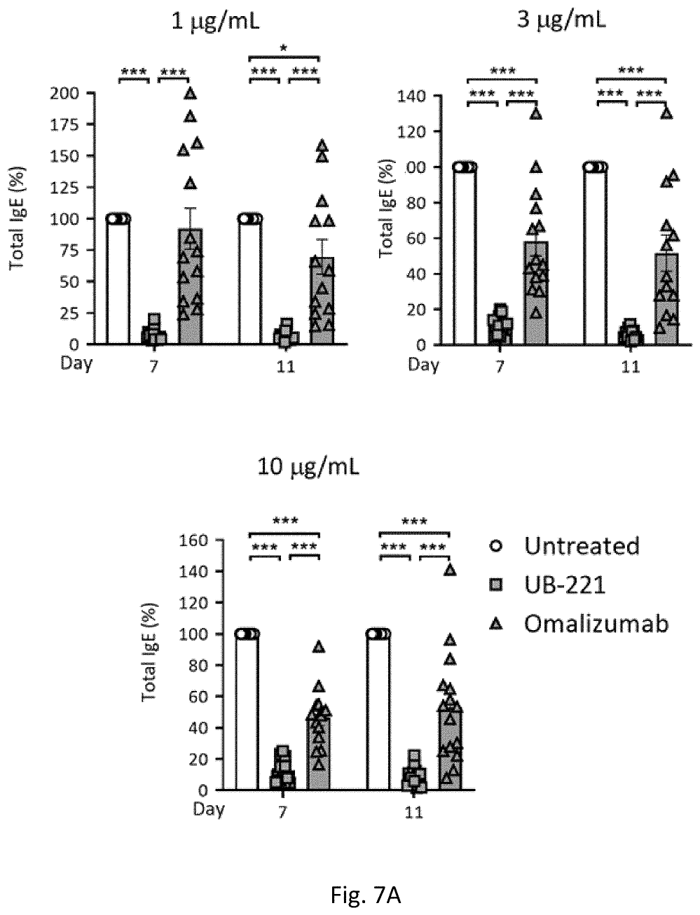

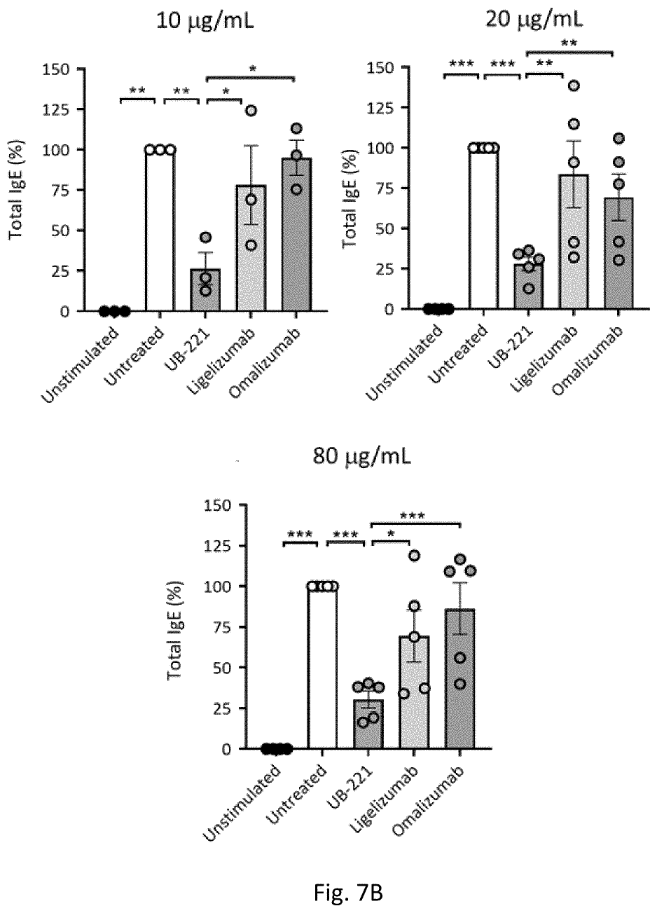

The results, shown in FIGS. 7A, indicate that UB-221 at all dose levels of 1, 3, and 10 μg/mL showed an overall 87-94% reduction of total IgE, which are superior over omalizumab as the latter reduced IgE at a lower rate of 7.9-53.5%. The additional study with doses at 10, 20, and 80 μg/mL (FIG. 7B) confirms the superiority of UB-221 over omalizumab, a greater 69%-74% versus a lower 4.9%-31% reduction. Ligelizumab reduced IgE by 16%-31% overall, with a trend of better reduction than omalizumab, though not statistically significant. Thus, UB-221 outperforms ligelizumab and omalizumab in CD23-mediated downregulation of IgE production, consistent with the finding (FIG. 6) that UB-221 in free form binds abundantly to CD23-occupied IgE and in IgE:mAb complex form UB-221 freely engages CD23, while ligelizumab reacts limitedly and omalizumab stays inert toward CD23

### Example 8: UB-221 can Bind Cynomolgus Macaque's IgE and CD23-Bound IgE and Induce a Rapid, Profound Serum IgE Reduction after a Single IV Dose

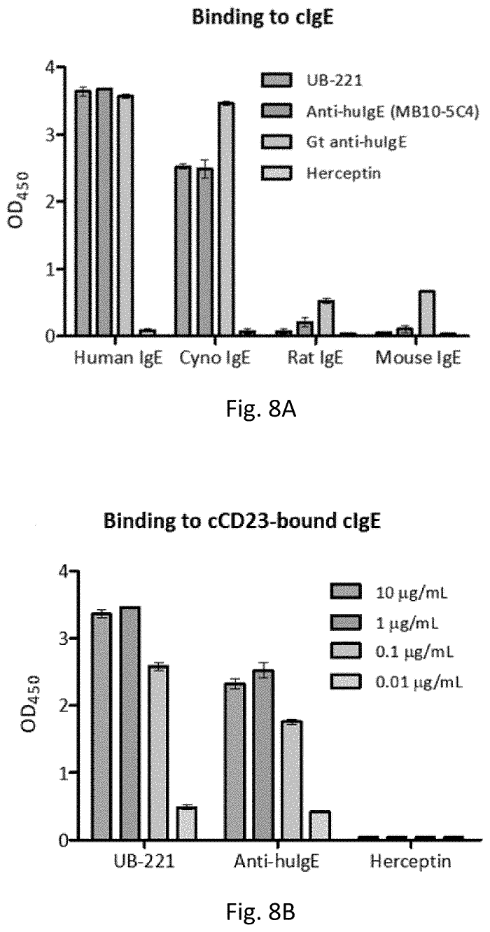

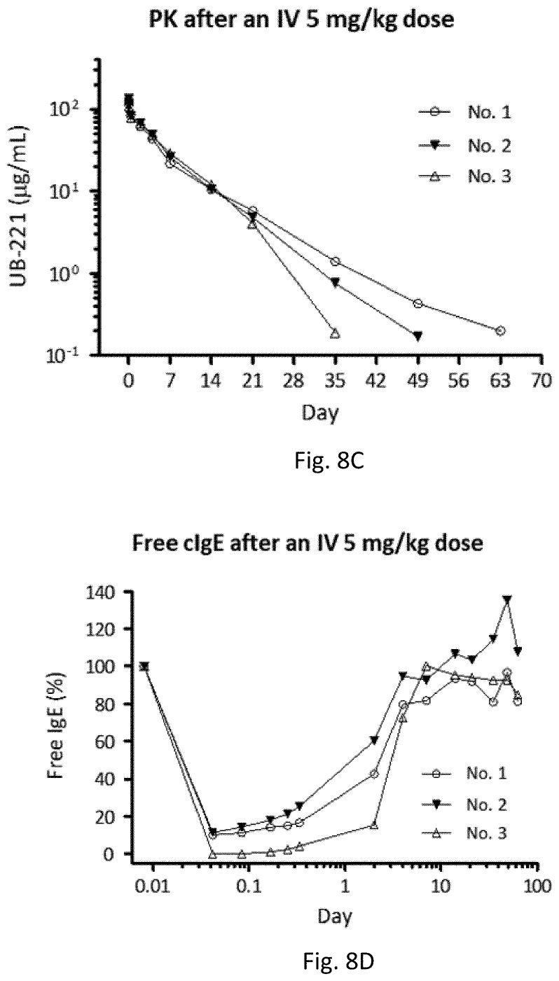

UB-221 can bind to cynomolgus macaques cIgE (FIG. 8A) and dose-dependently engage in cCD23-bound cIgE (FIG. 8B), proving that the cynomolgus can serve as an appropriate pharmacology and toxicology animal model. In cynomolgus (n=3) receiving a single IV dose of UB-221 at 5.0 mg/kg, the antibody declined in the serum with a mean elimination half-life of 6.3 days (FIG. 8C), in which UB-221 could induce a rapid, profound reduction of serum free cIgE by 90%-100% (FIG. 8D) from their basal levels around 400 ng/mL. The basal IgE levels were 434, 399 and 411 ng/mL for the macaques No. 1, 2, and 3, respectively.

### Example 9: Design of a First-In-Human Phase 1 Clinical Trial Demonstrating UB-221 is Safe and Well-Tolerated, and Capable of Inducing a Long-Lasting (3 to 6 Months) Disease-Improving Effect (UAS7 and HSS7 Scores) in Patients of Chronic Spontaneous Urticaria Treated with One Single IV Dose

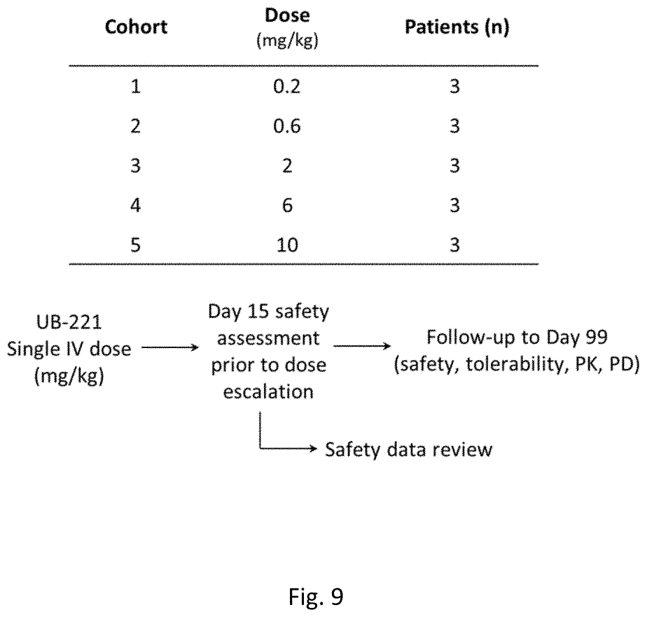

A phase-1, single-dose, dose-escalating of UB-221 by IV infusion as an add-on therapy was conducted to evaluate the safety, tolerability, pharmacokinetics (PK), and pharmacodynamics (PD) in chronic spontaneous urticaria (CSU) subjects under the first line H1-antihistamine treatment. A total of 15 eligible subjects were enrolled from two study sites in Taiwan and assigned to each of the 5 cohorts to receive a single IV-infusion dose of UB-221 at 0.2 (Cohort 1), 0.6 (Cohort 2), 2.0 (Cohort 3), 6.0 (Cohort 4) and 10 mg/kg (Cohort 5) (FIG. 9). All 15 enrolled subjects were Asian adults including 8 males and 7 females, with ages from 21 to 72 years old and weights from 49.0 to 87.6 kg. Two score systems were used to score the signs and symptoms of CSU: 1) UAS7 (Urticaria Activity Score) based scoring daily the number of daily wheals and itching intensity on a scale of 0 to 3 over 7 days; and 2) HSS7 (Hives Severity Score) based on scoring daily the number of hives on a scale of 0 to 3 over 7 days. Among 15 subjects, 9 subjects had severe CSU (UAS7 score 28-42), 4 subjects had moderate CSU (UAS7 score 16-27) and 2 subjects had mild CSU (UAS7 score 7-15) at baseline.

UB-221 is safe and well-tolerated. All 15 subjects completed the study. None of them discontinued treatment, withdrew, or lost to follow-up. Since no dose-limited toxicities were observed, the dose level of cohort 5 at 10 mg/kg was determined as the maximum tolerated dose. Most of the 39 treatment emergent adverse events (TEAE) were mild or moderate and unrelated to UB-221. Long-lasting suppression of disease symptoms for 2 months or longer was observed at and beyond doses of 2.0 mg/kg.

### Example 10: Serum Concentration Curves and Pharmacokinetic Profiles in Urticaria Patients after a Single IV Infusion of UB-221

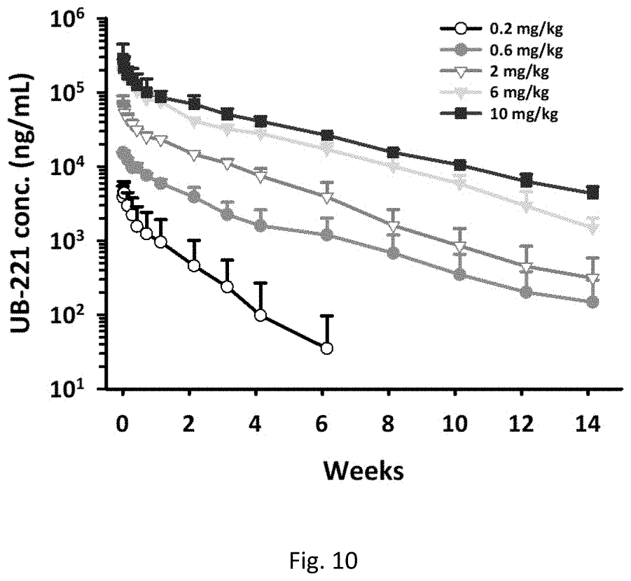

Serum concentrations of UB-221 following a single IV infusion dose were determined by ELISA using a neutralizing anti-UB-221 idiotypic monoclonal antibody which can specifically bind to UB-221 as the capture antibody coated on the plate. Mouse anti-human IgG Fc-HRP as the detection antibody. The results demonstrate a dose-dependent manner for the duration of UB-221 exposure. The half-life of UB-221 was estimated to be in the range of 16 to 22 days at doses of 0.6 to 10 mg/kg (FIG. 10).

### Example 11: Efficacy Score UAS in Urticaria Patients Treated with UB-221

11.1 Urticaria Activity Score Over 7 Days (UAS7)

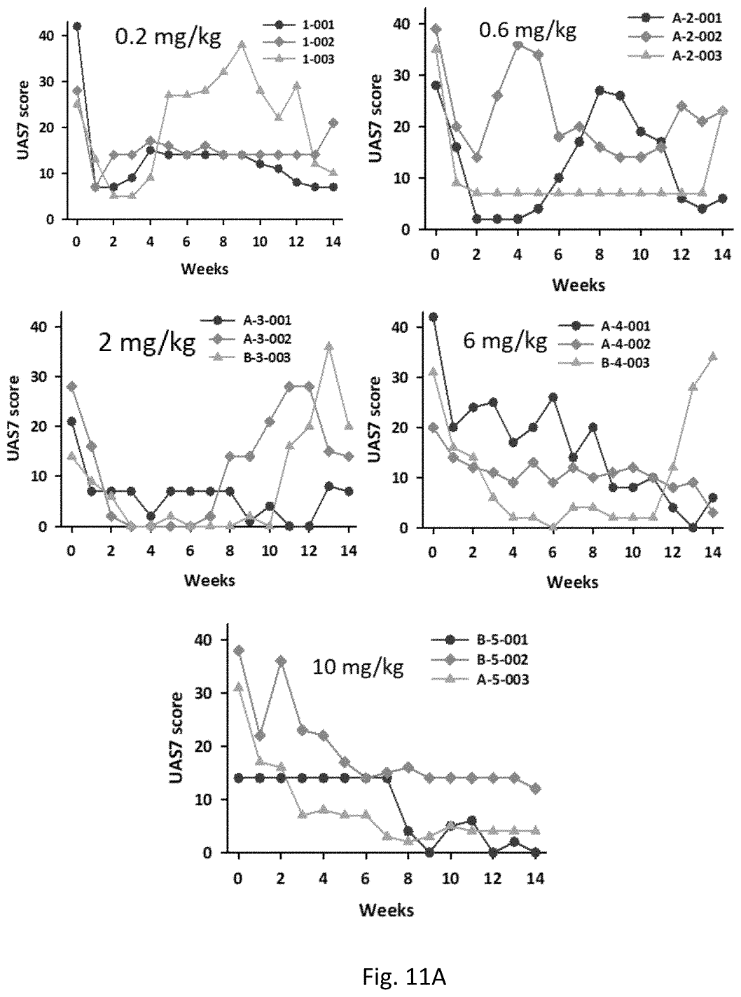

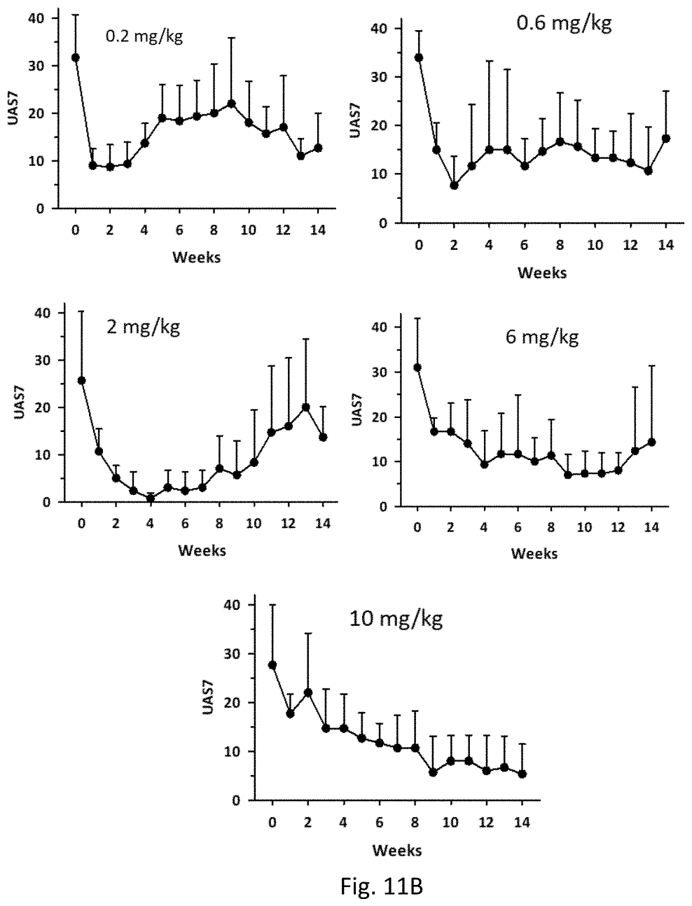

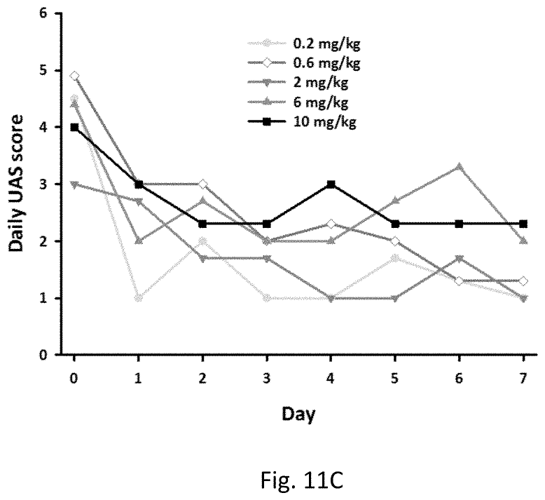

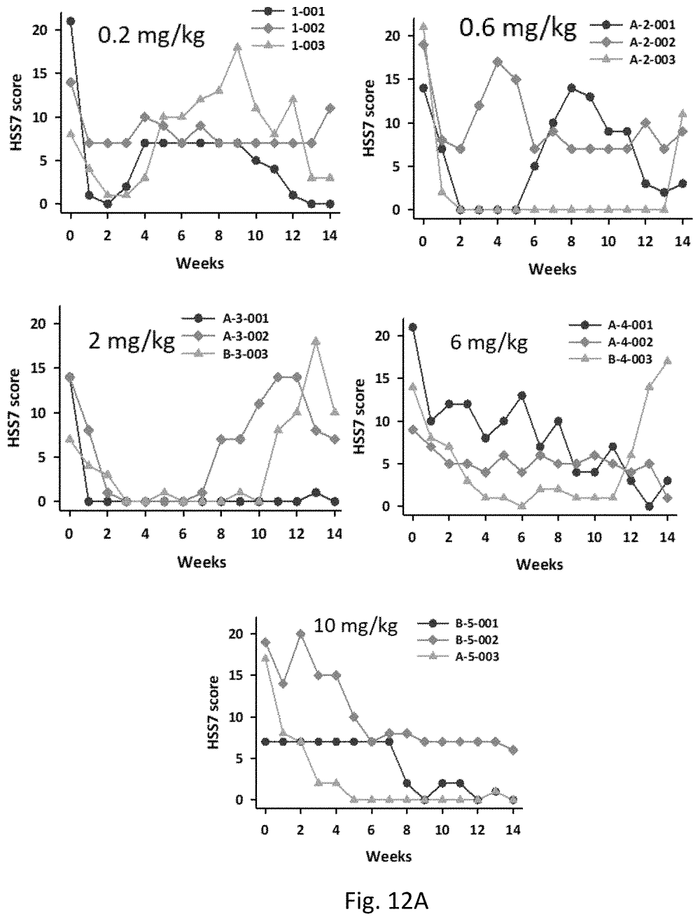

Another efficacy marker of UB-221 is evaluated by the relief of disease symptoms as the changes of UAS7 from baseline. The individual weekly change of disease scores UAS7 in each of 5 dose cohorts over 14 weeks is shown in FIG. 11A. The average (mean±SD) was calculated from 3 individual subjects (n=3) in each dose cohort (FIG. 11B). The higher scores of UAS7 indicate greater disease severity. The results show that in all 13 subjects with severe to moderate CSU (UAS7 score ≥16) at baseline, a rapid reduction of UAS7 occurred in the first week after receiving a single dose of UB-221 IV infusion. The subjects with milder CSU, such as one in Cohort 5 with a UAS7 score of 14 at baseline, had delayed response until 8 weeks after receiving a single dose of 10 mg/kg UB-221. The symptom relief to a well-controlled/disease-free stage (UAS7≤6) appeared dose-dependent and achieved in 10 out of 15 subjects (FIG. 11A). In particular, all 3 subjects in cohort 3 (2 mg/kg), all 3 subjects in cohort 4 (6 mg/kg) experienced a sustained disease-free stage, i.e., complete response (UAS7=0) for 2 to 14 weeks during the follow-up period. A single dose of UB-221 preserves the potential to achieve a mean score UAS7≤6 (disease under well control) (FIG. 11B), suggesting a single dose of >2 mg/kg can suppress the disease effectively for 3 to 6 months.

11.2 Daily Changes in UAS Disease Score

UAS scores from 3 subjects per cohort were collected daily and averaged. The daily mean values post infusion were compared to the baseline values, which were the mean daily UAS score from same subjects over 7 days prior to UB-221 infusion. Table 2 and FIG. 11C show the results.

| | Before infusion | | | | | | | |
|---|---|---|---|---|---|---|---|---|
| | (W −1) | Post UB-221 Infusion (W 1) | | | | | | |
| Cohort | Daily mean | Day 1 | Day 2 | Day 3 | Day 4 | Day 5 | Day 6 | Day 7 |
| 1 | 4.5 | 1.0 | 2.0 | 1.0 | 1.0 | 1.7 | 1.3 | 1.0 |
| 2 | 4.9 | 3.0 | 3.0 | 2.0 | 2.3 | 2.0 | 1.3 | 1.3 |
| 3 | 3.0 | 2.7 | 1.7 | 1.7 | 1.0 | 1.0 | 1.7 | 1.0 |
| 4 | 4.4 | 2.0 | 2.7 | 2.0 | 2.0 | 2.7 | 3.3 | 2.0 |
| 5 | 4.0 | 3.0 | 2.3 | 2.3 | 3.0 | 2.3 | 2.3 | 2.3 |

The profiles show that UAS disease scores declined rapidly on Day 1 and thereafter, suggesting that CSU patients experienced fast relief of symptoms within 24 hours of administering of UB-221.

### Example 12: Efficacy Score HSS7 in Urticaria Patients Treated with UB-221

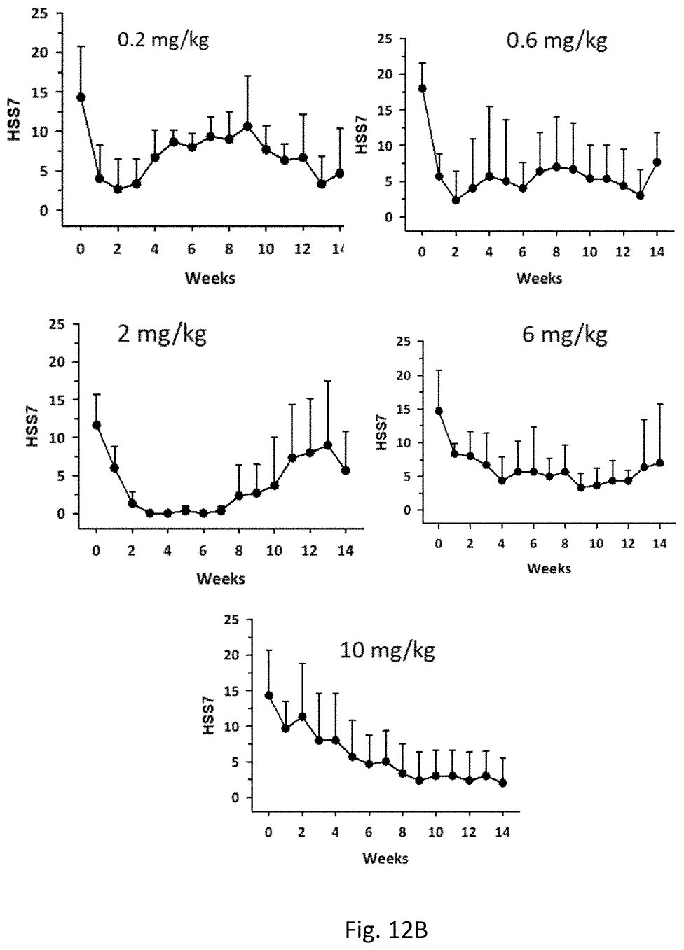

One efficacy marker of UB-221 is evaluated by the relief of disease symptoms as the changes of Hives Severity Score over 7 days (HSS7) from baseline. The individual weekly change of disease scores HSS7 in each of 5 dose cohorts over 14 weeks is shown in FIG. 12A. The average (mean±SD) was calculated from 3 individual subjects (n=3) in each dose cohort (FIG. 12B). The higher scores of HSS7 indicate greater disease severity. The results show that the HSS7 scores are parallel to the UAS7 scores (FIGS. 11A to 11C) in all subjects. A rapid reduction of HSS7 scores occurred in the first week after receiving a single IV infusion of UB-221. The symptom relief to well controlled/disease free stage (HSS7<6) appeared dose-dependent and achieved in 12 out of 15 subjects (FIG. 12A). In particular, 2 subjects in Cohort 1 (0.2 mg/kg), 2 subjects in Cohort 2 (0.6 mg/kg), all 3 subjects in Cohort 3 (2 mg/kg), all 3 subjects in Cohort 4 (6 mg/kg), and 2 subjects in Cohort 5 (10 mg/kg) experienced a sustained disease-free stage, i.e., complete response (HSS7=0) for 2 to 14 weeks during the follow-up period. Like the effect on HSS7 scores, a single dose UB-221 preserves a potential to achieve a mean score HSS7≤6 (disease under well control) (FIG. 12B), suggesting a single dose of >2 mg/kg can suppress the disease well for 3 to 6 months.

### Example 13: Serum Concentrations of Free IgE in Urticaria Patients Treated with UB-221

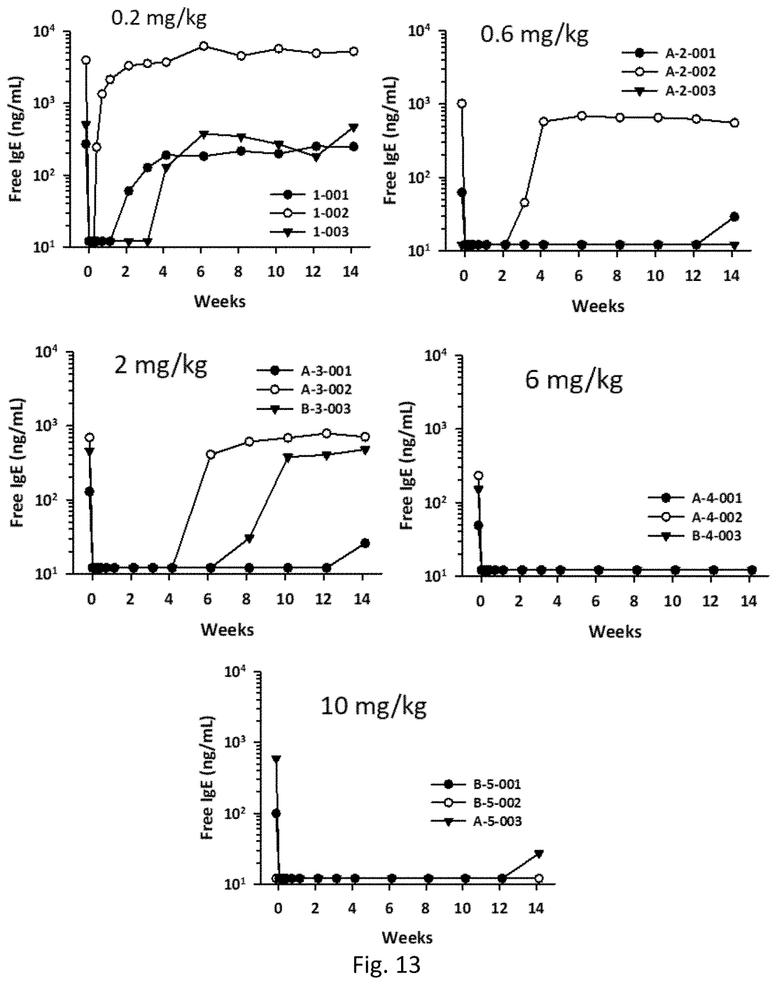

A single IV dose of UB-221 to urticaria patients can induce a rapid, profound reduction of serum free IgE (FIG. 13). Serum free IgE was determined by ELISA using recombinant FcεRIα-IgG.Fc fusion protein as a capture protein and the biotin-conjugated mouse anti-human IgE monoclonal antibody followed by addition of streptavidin-poly HRP for signal enhancement. The lower limit of quantification (LLOQ) for free IgE was 24 ng/mL. Before UB-221 infusion, the range of free IgE concentrations at baseline was 3999 to 272 ng/mL in cohort 1 (0.2 mg/kg), 1015 to <24 ng/mL in cohort 2 (0.6 mg/kg), 129 to 697 ng/mL in cohort 3 (2 mg/kg), 48.9 to 233 ng/mL in cohort 4 (6 mg/kg) and 593 to <24 ng/mL in cohort 5 (10 mg/kg). For all 15 subjects, serum IgE was completely neutralized by UB-221 (free IgE level <LLOQ) within 4 hours after UB-221 infusion. Complete IgE neutralization was sustained for 2 to 22 days in cohort 1 (0.2 mg/kg), 15 to 99 days in cohort 2 (0.6 mg/kg), 29 to 85 days in cohort 3 (2 mg/kg), >99 days in cohort 4 (6 mg/kg), and 85 to >99 days in cohort 5 (10 mg/kg). These data suggest that the efficacy of UB-221 is potent and long-lasting.

### Example 14. Concurrent Changes of Serum UB-211 Concentration, Serum Free IgE Level, and UAS7 Disease Score in Urticaria Patients after a Single IV Dose of UB-221

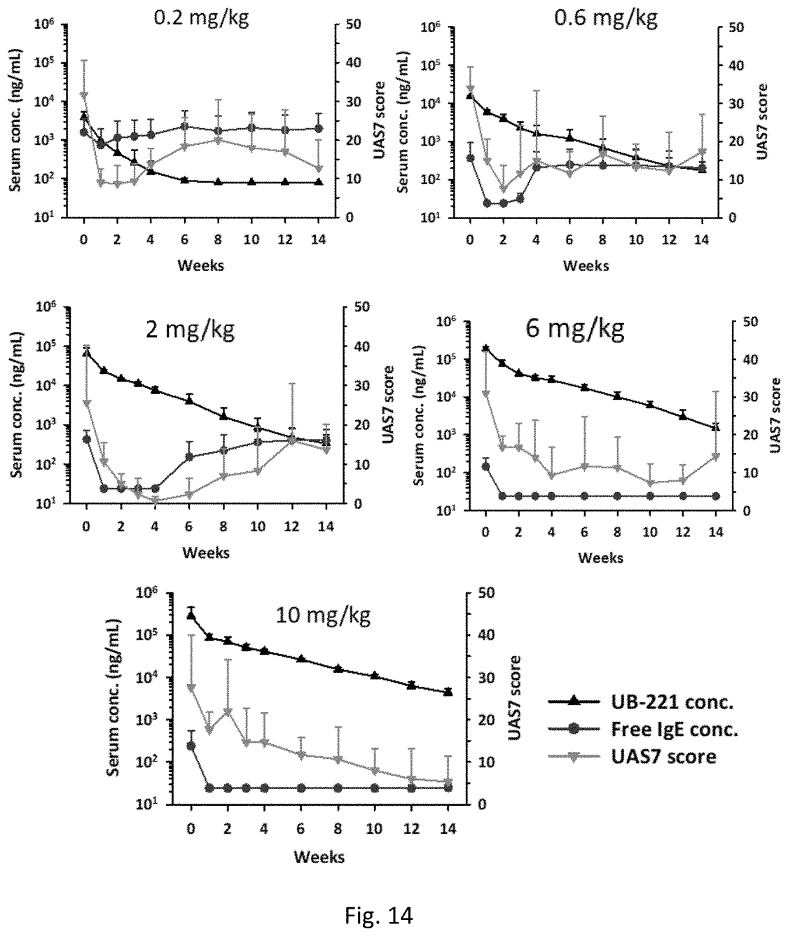

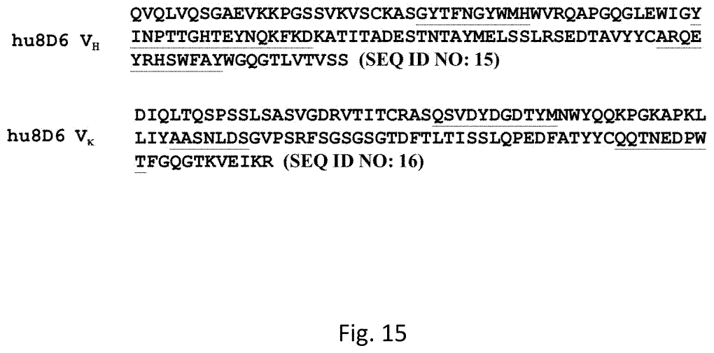

The averaged serum concentration of UB-221, serum free IgE level and UAS7 disease score are shown in parallel in subjects who participated in each of 5 dose cohorts over 14 weeks of the single-dose trial period. The average (mean) with SD was calculated from 3 individual subjects (n=3) in each cohort. There is a good correlation among these three parameters that at higher serum concentrations of UB-221, the serum free IgE levels were suppressed entirely and the reduction of UAS7 disease scores was maintained for a longer period (FIG. 14). The dose-dependent correlation suggests that a single IV dose of >2.0 mg/kg would potentially allow UB-221 to be administered every 3 to 6 months and achieve complete response (UAS7=0) or well controlled stage (UAS7<6) in the treatment of chronic spontaneous urticaria (CSU), a disease that the level of serum IgE plays a critical role.

### CONCLUSION

The present invention pertains to the finding of the UB-221 monoclonal antibody, a humanized anti-IgE IgG1 expressed in the CHO-S cell line, that can be potentially administered every 3 to 6 months to effectively treat the IgE-associated chronic spontaneous urticaria. This is demonstrated in the conduct of a single-dose phase 1 of UB-221 trial (Example 9) with supportive results described above (Examples 9 to 14). Such a every 3-month (12 weeks) or 6-month (24 weeks) less-frequent dose regimen presents a stark contrast with the other two anti-IgE mAbs, the only-approved omalizumab and the phase-3-trial ligelizumab failed to be developed further, that are administered every 4 weeks in the management of CSU²⁴.

The utility of a less-frequent dose regimen is due to UB-221's unique binding and functional profile compared to omalizumab and ligelizumab. UB-221 binds IgE with a strong affinity (Examples 1 and 2), performing superior over omalizumab in IgE neutralization (Example 3) and in the reduction of free IgE in sera of treated hIGHE-knockin mice (Example 4). UB-221 and ligelizumab neutralize high serum IgE in atopic dermatitis patients with equipotency, while omalizumab is less potent (Example 5). In cynomolgus macaques, a single dose of UB-221 can rapidly, profoundly bring down serum IgE levels (Example 8). These observations indicate that UB-221 performs as a potent IgE neutralizer.

UB-221 mAb is a newer-class of humanized anti-IgE IgG1 that differentiates it from omalizumab and ligelizumab in terms of the way interacting with CD23. UB-221 in free form binds to CD23-bound IgE and in IgE:mAb complex form engages CD23 in an unrestricted manner while both ligelizumab and omalizumab are limited in either way of indirect interaction with CD23 (Example 6), which correlates with the finding that UB-221 downregulates the greatest level of CD23-mediated IgE neo-synthesis in human PBMCs (Example 7). These observations indicate that UB-221 is a multifunctional anti-IgE antibody, in addition to a potent IgE neutralizer, which also performs as a significant IgE synthesis inhibitor, leading to an efficient IgE trapper 1) to mop up soluble free IgE and IgE:CD23 complex in circulation, 2) to form UB-221:IgE complex that binds CD23 to undergo CD23-dependent transcytosis and clearance²³) to remove IgE:allergen and IgE:autoantibody complexes by way of transport of UB-221:IgE complexes across the epithelial cells of gastrointestinal lumen and bronchoalveolar tracts. CD23 is expressed in numerous types of cells¹⁰. In contrast, omalizumab and ligelizumab mainly operate as plain IgE neutralizer.

The unique characteristics of UB-221 performing dual roles as a potent IgE neutralizer and a significant IgE synthesis inhibitor correlate with UB-221's potent IgE downregulation and its long-lasting disease-improving effect in the treatment with urticaria. The utility of less-frequent dose regimen, i.e., every 3 months (12 weeks) or 6 months (24 weeks), is thus can be applicable to a diverse array of allergic and non-allergic diseases that are IgE-mediated or IgE-associated.

### REFERENCES

1. Awankar R. Allergic diseases and asthma: a global public health concern and a call to action. World Allergy Organ J. 2014; 7:12. doi: 10.1186/1939-4551-7-12
2. Dierick B J H, van der Molen T, Flokstra-de Blok B M J, Muraro A, Postma M J, J W H Kocks, van Boven J F M. Burden and socioeconomics of asthma, allergic rhinitis, atopic dermatitis and food allergy. Expert Rev Pharmacoeconomics Outcomes Res. 2020; 20:437-453.
3. Bantz S K, Zhu Z, Zheng T. The atopic march: progression from atopic dermatitis to allergic rhinitis and asthma. J Clin Cell Immunol. 2014; 5:202. doi: 10.4172/2155-9899.1000202
4. Hill D A, Spergel J M. The atopic march—Critical evidence and clinical relevance. Ann Allergy Asthma Immunol. 2018; 120: 131-137.
5. Paller Amy S A S. The atopic march and atopic multimorbidity: many trajectories, many pathways. J Allergy Clin Immunol. 2019; 143:46-55.
6. Kinet J-P. The high-affinity IgE receptor (FcεRI): from physiology to pathology. Annu Rev Immunol 1999; 17:931-972.
7. Conrad D H. FcεRII/CD23: the low affinity receptor for IgE. Annu Rev Immunol 1990; 8:623-645.
8. Gould H J, Sutton B J. IgE in allergy and asthma today. Nat Rev Immunol 2008; 8(3):205-217.
9. Sutton B J, Davies A M. Structure and dynamics of IgE-receptor interaction:FcεRI and CD23/FceRII. Immnol. Rev. 2015; 268:222-235.
10. Acharya, M. et al. CD23/FcεRII: molecular multi-tasking. Clin. Exp. Immunol. 2010; 162:12-23.
11. Conrad, D. H., Ford, J. W., Sturgill, J. L. & Gibb, D. R. CD23: an overlooked regulator of allergic disease. Curr. Allergy Asthma Rep. 2007; 7:331-337.
12. Kelly A E, Chen B-H, Woodward E C, Conrad D H. Production of a chimeric form of CD23 that is oligomeric and blocks IgE binding to the FcεRI. J Immunol 1998; 161:6696-6704.
13. Schwartz C, Turqueti-Neves A, Hartmann S, Yu P, Nimmerjahn F, Voehringer D. Basophil-mediated protection against gastrointestinal helminths requires IgE-induced cytokine secretion. Proc Natl Acad Sci USA 2014; 111:E5169-5177.
14. Ferastraoaru D, Jordakieva G, Jensen-Jarolim E. The other side of the coin: IgE deficiency, a susceptibility factor for malignancy occurrence. World Allergy Organization J. 2021; 14:100505. http://doi.org/10.1016/j.waojou.2020.100505
15. Xolair FDA Approval History. Drug.com https://www.drugs.com/history/xolair.html
16. Guntern P, Eggel A. Past, present, and future of anti-IgE biologics. Allergy 2020; 75:2491-2502.
17. Balbino B, Conde E, Marichal T, Starkl P, Reber L L. Approaches to target IgE antibodies in allergic diseases. Pharmacol. Ther. 2018; 191:50-64.
18. Pharmacokinetics, pharmacodynamics and safety of QGE031 (ligelizumab), a novel high-affinity anti-IgE antibody, in atopic subjects. Clin Exp Allergy 2014; 44:1371-1385.
19. Maurer M, Giménez-Arnau A M, Sussman G, Metz M, Baker D R, et al. Ligelizumab for chronic spontaneous urticaria. New Engl J Med 2019; 381:1321-1332.
20. Gasser P, Tarchevkaya S S, Guntern P, Brigger D, Ruppli R, et al. The mechanistic and functional profile of the therapeutic anti-IgE antibody ligelizumab differs from omalizumab. Nat Communication 2020; 11:165 DOI: 10.1038/s41467-019-13815-w
21. Chen J-B, Ramadani F, Pang M O Y, Rebecca L. Beavil R L, et al. Structural basis for selective inhibition of immunoglobulin E-receptor interactions by an anti-IgE antibody. Sci Rep. 2018; 8:11548. DOI:10.1038/s41598-018-29664-4.
22. Shiung Y-Y, Chiang C-Y, Chen J-B, Wu P C, Hung A F-H, et al. An anti-IgE monoclonal antibody that binds to IgE on CD23 but not on high-affinity IgE.Fc receptors. Immunobiol. 2012; 217:676-683.
23. Tu Y, Salim S, Bourgeois J, et al. CD23-Mediated IgE Transport Across Human Intestinal Epithelium: Inhibition by Blocking Sites of Translation or Binding”, Gasgtroenterol. 2005; 129:928-940.
24. Maurer M, Eggel A, Bernstein J A, Gimenez A M, Soong W. Breakthrough in the understanding of the anti-IgE pathway in chronic spontaneous urticaria and the potential of ligelizumab. EMJ Allerg Immuno, 2020; 5:20-27.

1. A method for treating an IgE-mediated disease, comprising administering to a subject in need thereof an anti-IgE antibody, wherein the antibody is a multifunctional antibody against IgE which neutralizes IgE and inhibits IgE synthesis.

2. The method of claim 1, wherein the antibody is administered in an amount effective in providing a rapid and/or sustained symptomatic relief of the IgE-mediated disease.

3. The method of claim 1, wherein the antibody binds to free IgE, membrane-bound IgE on B lymphocytes, and/or IgE bound by CD23, but not to IgE bound by FcεRI on mast cells.

4. The method of claim 1, wherein the antibody binds in a free form with CD23-bound IgE and in an IgE-complex form with CD23.

5. The method of claim 1, wherein the antibody is an antigen-binding fragment thereof.

6. The method of claim 1, wherein the antibody is humanized.

7. The method of claim 1, wherein the antibody or antigen-binding fragment comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13.

8. The method of claim 7, wherein
the VH comprises the amino acid sequence of SEQ ID NO: 15; and/or
the VL comprises the amino acid sequence of SEQ ID NO: 16.

9. The method of claim 2, wherein symptomatic relief includes IgE reduction, alleviation in itch and/or decrease of hive counts.

10. The method of claim 2, wherein the symptomatic relief is sustained for 2 to 14 weeks or longer after administration.

11. The method of claim 2, wherein the symptomatic relief occurs within one week or faster after administration.

12. The method of claim 1, wherein the antibody is administered every 2 to 14 weeks or less frequently.

13. The method of claim 1, wherein the antibody is administered every 4 weeks or less frequently.

14. The method of claim 1, wherein the antibody is administered every 12 to 24 weeks.

15. The method of claim 1, wherein the antibody is administered at a dosage of 0.1 to 10 mg per 1 kilogram of body weight of the subject.

16. The method of claim 1, wherein the antibody is included in a composition in an entire dose and is administered to the subject in a single dose.

17. The method of claim 1, wherein the antibody is administered by either intravenous or subcutaneous injection.

18. The method of claim 1, wherein the IgE-mediated disease is allergic asthma, allergic rhinitis, atopic dermatitis, food allergy, chronic spontaneous (idiopathic) urticaria, chronic rhinosinusitis, systemic mastocytosis, cutaneous mastocytosis, allergic bronchopulmonary aspergillosis, recurrent idiopathic angioedema, or eosinophil-associated gastrointestinal disorder.

19. An anti-IgE antibody for use in treating an IgE mediated disease in a subject in need thereof wherein the antibody is a multifunctional antibody against IgE which neutralizes free IgE and inhibits IgE synthesis.

20. The anti-IgE antibody for use according to claim 19, which provides a rapid and/or sustained symptomatic relief in the subject.

21. The anti-IgE antibody for use according to claim 19, wherein the antibody binds to free IgE, membrane-bound IgE on B lymphocytes, or IgE bound by CD23, but not to IgE bound by FcεRI on mast cells.

22. The anti-IgE antibody for use according to claim 19, wherein the antibody binds in a free form with CD23-bound IgE and in an IgE-complex form with CD23.

23. The anti-IgE antibody for use according to claim 19, wherein the antibody is an antigen-binding fragment thereof.

24. The anti-IgE antibody for use according to claim 19, wherein the antibody is humanized.

25. The anti-IgE antibody for use according to claim 19, wherein the antibody or antigen-binding fragment comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13.

26. The anti-IgE antibody for use according to claim 25, wherein
the V_{H} comprises the amino acid sequence of SEQ ID NO: 15; and/or
the V_{L} comprises the amino acid sequence of SEQ ID NO: 16.

27. The anti-IgE antibody for use according to claim 20, wherein symptomatic relief includes IgE reduction, alleviation in itch and/or decrease of hive counts.

28. The anti-IgE antibody for use according to claim 20, wherein the symptomatic relief is sustained for 2 to 14 weeks or longer after administration.

29. The anti-IgE antibody for use according to claim 20, wherein the symptomatic relief occurs within one week or faster after administration.

30. The anti-IgE antibody for use according to claim 19, wherein the antibody is administered every 2 to 14 weeks or less frequently.

31. The anti-IgE antibody for use according to claim 19, wherein the antibody is administered every 4 weeks or less frequently.

32. The anti-IgE antibody for use according to claim 19, wherein the antibody is administered every 12 to 24 weeks.

33. The anti-IgE antibody for use according to claim 19, wherein the antibody is administered at a dosage of 0.1 to 10 mg per 1 kilogram of body weight of the subject.

34. The anti-IgE antibody for use according to claim 19, wherein the antibody is included in a composition in an entire dose and is administered to the subject in a single dose.

35. The anti-IgE antibody for use according to claim 19, wherein the antibody is administered by either intravenous or subcutaneous injection.

36. The anti-IgE antibody for use according to claim 19, wherein the IgE-mediated disease is allergic asthma, allergic rhinitis, atopic dermatitis, food allergy, chronic spontaneous (idiopathic) urticaria, chronic rhinosinusitis, systemic mastocytosis, cutaneous mastocytosis, allergic bronchopulmonary aspergillosis, recurrent idiopathic angioedema, or eosinophil-associated gastrointestinal disorder.

37-42. (canceled)

43. A pharmaceutical composition for use in treating an IgE-mediated disease comprising an anti-IgE antibody and a pharmaceutically acceptable excipient, wherein the antibody is a multifunctional antibody against IgE, which neutralizes free IgE and inhibits IgE synthesis.

44. The pharmaceutical composition of claim 43, which is effective in providing rapid and/or sustained symptomatic relief in a subject in need thereof.

45. The pharmaceutical composition of claim 43, wherein
the antibody binds to free IgE, membrane-bound IgE on B lymphocytes, and/or IgE bound by CD23, but not to IgE bound by FcεRI on mast cells;
the antibody binds in a free form with CD23-bound IgE and in an IgE-complex form with CD23;
the antibody is an antigen-binding fragment thereof;
the antibody is humanized; and/or
the antibody or antigen-binding fragment comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13;
preferably the VH comprises the amino acid sequence of SEQ ID NO: 15; and/or
preferably the VL comprises the amino acid sequence of SEQ ID NO: 16.

46. The pharmaceutical composition of claim 44, wherein
the symptomatic relief includes IgE reduction, alleviation in itch and/or decrease of hive counts;
the symptomatic relief is sustained for 2 to 14 weeks or longer after administration; and/or
the symptomatic relief occurs within one week or faster after administration.

47. The pharmaceutical composition of claim 43, wherein
the antibody is administered every 2 to 14 weeks or less frequently;
the antibody is administered every 4 weeks or less frequently;
the antibody is administered every 12 to 24 weeks;
the antibody is administered in a dosage of 0.1 to 10 mg per 1 kilogram of body weight of the subject;
the antibody is included in a composition in an entire dose and is administered to the subject in a single dose; and/or
the antibody is administered by either intravenous or subcutaneous injection.

48. The pharmaceutical composition of claim 43, wherein
the IgE-mediated disease is allergic asthma, allergic rhinitis, atopic dermatitis, food allergy, chronic spontaneous (idiopathic) urticaria, chronic rhinosinusitis, systemic mastocytosis, cutaneous mastocytosis, allergic bronchopulmonary aspergillosis, recurrent idiopathic angioedema, or eosinophil-associated gastrointestinal disorder.

49. (canceled)
